SEQUENCE LISTING (40 sequences)

SEQ ID NO: 1 - DNA, 27 bp, Artificial Sequence
  Synthetic Construct
agaattttgccacctctctttattcca

SEQ ID NO: 2 - DNA, 27 bp, Artificial Sequence
  Synthetic Construct
ccacttccacggcctgaggtggtttct

SEQ ID NO: 3 - DNA, 23 bp, Artificial Sequence
  Synthetic Construct
tccattccgagcatccgaagact

SEQ ID NO: 4 - DNA, 23 bp, Artificial Sequence
  Synthetic Construct
gtggtgctgcttgcgctgccggt

SEQ ID NO: 5 - DNA, 27 bp, Artificial Sequence
  Synthetic Construct
accttctttccagtgctaaaggatctc

SEQ ID NO: 6 - DNA, 26 bp, Artificial Sequence
  Synthetic Construct
tcagcttcacattctcaggaactctc

SEQ ID NO: 7 - DNA, 28 bp, Artificial Sequence
  Synthetic Construct
gccatggctctcagagtccttctgttaa

SEQ ID NO: 8 - DNA, 26 bp, Artificial Sequence
  Synthetic Construct
atgtccccatgctcctgcgccagcaa

SEQ ID NO: 9 - DNA, 30 bp, Artificial Sequence
  Synthetic Construct
ggcccagggagaacggggaagggacattta

SEQ ID NO: 10 - DNA, 28 bp, Artificial Sequence
  Synthetic Construct
actttagaaagaacacattccatgaaag

SEQ ID NO: 11 - DNA, 22 bp, Artificial Sequence
  Synthetic Construct
ggcccaacgccagatcaagcgg

SEQ ID NO: 12 - DNA, 27 bp, Artificial Sequence
  Synthetic Construct
ctaacccaagtcctggaggttgttatg

SEQ ID NO: 13 - DNA, 23 bp, Artificial Sequence
  Synthetic Construct
gcagcgtgcagatcaacttcttc

SEQ ID NO: 14 - DNA, 28 bp, Artificial Sequence
  Synthetic Construct
ctttatcaagcatcagccatgatggtag

SEQ ID NO: 15 - DNA, 27 bp, Artificial Sequence
  Synthetic Construct
accctgctccacgatgcggtaatgaat

SEQ ID NO: 16 - DNA, 22 bp, Artificial Sequence
  Synthetic Construct
tggcaggcttcagcttcgtcag

SEQ ID NO: 17 - DNA, 27 bp, Artificial Sequence
  Synthetic Construct
gcagtcaactggagagagttccgattt

SEQ ID NO: 18 - DNA, 29 bp, Artificial Sequence
  Synthetic Construct
ctccttatttttgtttctggaaaagttgt

SEQ ID NO: 19 - DNA, 28 bp, Artificial Sequence
  Synthetic Construct
tattggtcaaagaaactggttacttttt

SEQ ID NO: 20 - DNA, 29 bp, Artificial Sequence
  Synthetic Construct
cgctacctgcagcgccacgtgaagctcat

SEQ ID NO: 21 - DNA, 27 bp, Artificial Sequence
  Synthetic Construct
gactataagaaagctcttctgctgctc

SEQ ID NO: 22 - DNA, 30 bp, Artificial Sequence
  Synthetic Construct
ttactgggcatcagtagaagaatatattcc

SEQ ID NO: 23 - DNA, 28 bp, Artificial Sequence
  Synthetic Construct
cagttcaaatggcaaactggaagaagtg

SEQ ID NO: 24 - DNA, 27 bp, Artificial Sequence
  Synthetic Construct
cagaatggcttccatttgccaggccta

SEQ ID NO: 25 - DNA, 22 bp, Artificial Sequence
  Synthetic Construct
gaacaacaccctggacatggct

SEQ ID NO: 26 - DNA, 26 bp, Artificial Sequence
  Synthetic Construct
tctggctgaagttgaggatctcttac

SEQ ID NO: 27 - DNA, 25 bp, Artificial Sequence
  Synthetic Construct
gttcaacttggacactgaaaacgca

SEQ ID NO: 28 - DNA, 26 bp, Artificial Sequence
  Synthetic Construct
atgttctctggatgtcagctgagtca

SEQ ID NO: 29 - DNA, 27 bp, Artificial Sequence
  Synthetic Construct
tgagataggatcatgaaggaagaggtg

SEQ ID NO: 30 - DNA, 24 bp, Artificial Sequence
  Synthetic Construct
aaagctggtcaaaagtgcaagcct

SEQ ID NO: 31 - DNA, 26 bp, Artificial Sequence
  Synthetic Construct
tcgtccatccgcaacgcgcacagcat

SEQ ID NO: 32 - DNA, 27 bp, Artificial Sequence
  Synthetic Construct
tggcagatatctacctagcatctcagt

SEQ ID NO: 33 - DNA, 23 bp, Artificial Sequence
  Synthetic Construct
acaccaagctcatcttgtgccca

SEQ ID NO: 34 - DNA, 26 bp, Artificial Sequence
  Synthetic Construct
tgcactgcttattgtagacagtgcca

SEQ ID NO: 35 - DNA, 22 bp, Artificial Sequence
  Synthetic Construct
tgcagatgtacaactcgcagca

SEQ ID NO: 36 - DNA, 26 bp, Artificial Sequence
  Synthetic Construct
gctgtgatgtgaaaaccacgtttgtc

SEQ ID NO: 37 - DNA, 24 bp, Artificial Sequence
  Synthetic Construct
gaccctgatcttgtgtacagccca

SEQ ID NO: 38 - DNA, 28 bp, Artificial Sequence
  Synthetic Construct
ctaaagtcagagcagaaatttatgaagc

SEQ ID NO: 39 - DNA, 25 bp, Artificial Sequence
  Synthetic Construct
tgataagacctacgccatgggacat

SEQ ID NO: 40 - DNA, 27 bp, Artificial Sequence
  Synthetic Construct
tgtgacgaatgtggacaaaccttcaag

(19) United States
(12) Patent Application Publication
(10) Pub. No.: US 2020/0291477 A1
(43) Pub. Date: Sep. 17, 2020

(54) ASSAYS FOR DETECTION OF ACUTE LYME DISEASE

(71) Applicants: The Regents of the University of California, Oakland, CA (US); The Johns Hopkins University, Baltimore, MD (US)

(72) Inventors: Charles Y. CHIU, San Francisco, CA (US); Jerome BOUQUET, San Francisco, CA (US); Mark J. SOLOSKI, Baltimore, MD (US); John N. AUCOTT, Lutherville, MD (US)

(21) Appl. No.: 16/767,098

(22) PCT Filed: Nov. 27, 2018
(86) PCT No.: PCT/US2018/062639

§ 371 Date: May 26, 2020

Related U.S. Application Data
(60) Provisional application No. 62/591,660, filed on Nov. 28, 2017.

(51) Int. Cl. C12Q 1/6883; C12N 15/10; C12Q 1/6874; G16B 25/10; G16B 20/00; G16B 40/00; A61K 9/00; A61K 31/65; A61K 31/43; A61K 31/546; A61P 31/04; G16H 10/40; G16H 70/60; G16H 50/20; G16H 20/10

(52) U.S. Cl. CPC C12Q 1/6883; C12Q 2600/158; C12N 15/1093; C12Q 1/6874; G16B 25/10; G16B 20/00; G16B 40/00; A61K 9/0053; A61K 31/65; A61K 31/43; A61K 31/546; A61P 31/04; G16H 10/40; G16H 70/60; G16H 50/20; G16H 20/10; C12N 15/1003

(57) ABSTRACT

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample from the subject.

### CROSS-REFERENCE TO RELATED APPLICATIONS

This application claims the benefit of U.S. Provisional Application No. 62/591,660, filed Nov. 28, 2017, which is hereby incorporated by reference in its entirety.

### STATEMENT REGARDING FEDERALLY SPONSORED RESEARCH OR DEVELOPMENT

This invention was made with government support under Grant Nos. R01 HL105704 and P30 AR053503 awarded by the National Institutes of Health. The government has certain rights in the invention.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 643662002140SEQLIST.TXT, date recorded: Nov. 27, 2018, size: 8 KB).

### TECHNICAL FIELD

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample from the subject.

### BACKGROUND

Lyme disease is a systemic tick-borne infection caused by Borrelia burgdorferi, and it is the most common vector-borne disease in the United States and Europe (Stanek et al., The Lancet, 379:461-473, 2012). Over 30,000 cases of Lyme disease are reported annually in the United States to the Centers for Disease Control and Prevention (see, e.g., CDC Lyme Disease Data and Statistics webpage). It is thought, however, that Lyme disease is under-reported due to inadequate diagnostic testing, and therefore the actual prevalence of Lyme disease has been estimated to be at least ten times higher (Hinckley et al., Clin Infect Dis, 59:676-681, 2014). If left undiagnosed and thus untreated, Lyme disease can cause arthritis, facial palsy, neuroborreliosis (neurological disease caused by B. burgdorferi that can include meningitis, radiculopathy, and occasionally encephalitis), and even myocarditis resulting in sudden death (see, e.g., CDC Lyme Disease Signs and Symptoms webpage). Most patients (80-90%) treated with appropriate antibiotics recover rapidly and completely, but 10-20% of patients develop persistent or recurring symptoms. When treated patients develop prolonged symptoms, these patients are considered to have post-treatment Lyme disease syndrome (Aucott et al., Int J Infect Dis, 17:e443-e449). The length of recovery time from Lyme disease is linked to the timing of diagnosis and treatment. The longer Lyme disease remains undiagnosed and untreated, the longer recovery time will be (Margues, Infect Dis Clin North Am, 22:341-360, 2008).

Despite the advantages of early diagnosis and treatment, diagnosing Lyme disease at an early stage of disease development remains challenging. One reason for this is because clinical manifestations can be highly variable. Often, patients present with non-specific “flu-like” symptoms early in the course of the illness, and without a history of tick bite. The classic erythema migrans (EM) “bullseye” rash is seen in fewer than 70% of patients. The majority of individuals show either uniformly red skin lesions that can be mistaken for other skin conditions, or no skin lesions at all (Steere and Sikand, N Engl J Med, 348:2472-2474, 2003). Moreover, current diagnostic tests are only effective at a later stage of disease development or unable to reliably detect Lyme disease. The standard method is serological testing, and the CDC recommends a two-tier serological assay for Lyme disease diagnosis. Serological testing, however, misses the window of early acute infection and can be negative in up to 40% of early acute cases (Steere et al., Clin Infect Dis, 47:188-195, 2008). Another diagnostic option, nucleic acid testing, is hindered by low titers of B. burgdorferi in the blood during acute infection, and has a reported sensitivity of detection of only 20-62% (Aguero-Rosenfeld et al., Clin Microbiol Reg, 18:484-509, 2005; and Eshoo et al., PLoS One, 7:e36825, 2012). As such, clinicians from regions endemic for Lyme disease often make diagnoses on the basis of patient clinical presentation and history. Diagnoses based solely on clinical presentation result in some patients being inappropriately treated for Lyme disease, while other patients are not treated in a timely fashion. Ultimately, the failure to accurately diagnose Lyme disease due to the absence of a sensitive and specific test can lead to devastating outcomes, including sudden cardiac death from Lyme carditis (Forrester et al., MMWR, 63:982-983, 2014).

Thus, there exists a need for methods to specifically detect Lyme disease at the early acute stage in order to provide appropriate and timely treatment.

### SUMMARY

The present disclosure relates to measuring gene expression of cells of a blood sample obtained from a mammalian subject suspected of having a tick-borne disease. In particular, the present disclosure provides tools for determining whether a human subject has acute Lyme disease by transcriptome profiling a peripheral blood mononuclear cell sample from the subject.

The present disclosure provides methods for measuring gene expression, comprising the steps of: (a) measuring RNA expression of a plurality of genes of cells from a blood sample obtained from a mammalian subject suspected of having a tick-borne disease; (b) calculating a weighted RNA expression score for each of the plurality of genes; and (c) calculating a Lyme disease score by taking the sum of the weighted RNA expression scores. In some embodiments, the mammalian subject is a human. In some embodiments, the methods are for providing information to assess whether a subject has acute Lyme disease. In some embodiments, the methods further comprise: step (d) identifying the subject as not having acute Lyme disease when the Lyme disease score is negative; or identifying the subject as having acute Lyme disease when the Lyme disease score is positive. In some embodiments, the methods further comprise one or more steps before step (a), which are selected from the group consisting of: obtaining a blood sample from the subject; isolating peripheral blood mononuclear cells (PBMCs) from the blood sample; and extracting RNA from the PBMCs. In some embodiments, the blood sample is whole blood. In some embodiments, the plurality of genes comprises at least 3, 4, 5, 6, 7, 8, 9, 10, 11, 12, 13, 14, 15, 16, 17, 18, 19 or all 20 genes of the group consisting of ANXA5, C3orf14, CDCA2, CR1, GBP2, IF127, ITGAM, KCNJ2, KIF4A, MLF1IP, NCF1, PLBD1, PLK1, RAD51, SLC25A37, STAB1, STEAP4, TBP, TNFSF13B, and ZNF276. In some embodiments, the plurality of genes comprises 1, 2, 3, 4 or all 5 genes of the group consisting of NCF1, ANXA5, CR1, STAB1, and MLF1IP. In some embodiments, step (a) comprises one or more of the group consisting of sequence analysis, hybridization, and amplification. In some preferred embodiments, step (a) comprises targeted RNA expression resequencing comprising: (i) preparing an RNA expression library for the plurality of targeted genes from RNA extracted from the PBMCs; (ii) sequencing a portion of at least 50,000 members of the library; and (iii) generating a read count for RNA expression of the plurality of genes by normalization to the sequence of the at least 50,000 members of step (ii). In some embodiments, step (a) comprises whole transcriptome shotgun sequencing (WTSS) comprising: (i) preparing an RNA expression library for the plurality of genes from RNA extracted from the PBMCs; (ii) sequencing a portion of at least 1,000,000 members of the library; and (iii) generating a read count for RNA expression of the plurality of genes by normalization to the sequence of the at least 1,000,000 members of step (ii). In some embodiments, step (b) comprises: multiplying the read count for each of the plurality of genes by a predetermined gene expression weight to obtain the weighted RNA expression score. In some embodiments, step (a) comprises: performing reverse transcriptase-quantitative polymerase chain reaction (RT-qPCR) on RNA extracted from the PBMCs. In other embodiments, step (a) comprises: hybridizing RNA extracted from the PBMCs to a microarray. In further embodiments, step (a) comprises: performing serial amplification of gene expression (SAGE) on RNA extracted from the PBMCs.

Furthermore, the present disclosure provides variations on the methods of the preceding paragraph. In some embodiments, the subject was bitten by a tick in a region where at least 20% of ticks are suspected of being infected with Borrelia burgdorferi. In some embodiments, the subject was bitten by a tick within three weeks of the blood sample being obtained. In some preferred embodiments, the subject has an erythema migrans rash when the blood sample was obtained, while in other preferred embodiments, the subject does not have an erythema migrans rash when the blood sample was obtained. In some embodiments, the subject has flu-like symptoms when the blood sample was obtained. Also, in some embodiments the methods further comprise performing a serologic test for Lyme disease. In some embodiments, the subject was determined to be negative for Lyme disease by serologic testing (either at the time the blood sample was obtained or within one or two weeks of the blood sample being obtained. In some embodiments, the methods further comprising performing a metabolomic or proteomic test for Lyme disease. In some embodiments, the tick-borne disease the subject is suspected of having is selected from the group consisting of Borreliosis (e.g., Lyme disease), Southern tick associated rash illness, Q fever, Colorado tick fever, Powassan virus infection, tick-borne encephalitis virus infection, tick-borne relapsing fever, Heartland virus infection and severe fever with thrombocytopenia virus infection. In some preferred embodiments, the tick-borne disease the subject is suspected of having is Borreliosis. In some embodiments, the Borreliosis is associated with infection with a Borrelia species selected from the group consisting of B. burgdorferi, B. azelli, and B. garinii. In some embodiments, the tick-borne disease the subject is suspected of having is selected from the group consisting of Anaplasmosis, Babesiosis, Ehrlichiosis, Lyme disease, Rickettsiosis, and Tularemia. In some embodiments, in which the subject was identified as having acute Lyme disease (e.g., when the Lyme disease score is positive), the methods further comprise: step (e) administering an antibiotic therapy to the subject to treat the Lyme disease. In some embodiments, the antibiotic therapy comprises an effective amount of an antibiotic selected from the group consisting of tetracyclines, penicillins, and cephalosporins. In some embodiments, the antibiotic therapy comprises an effective amount of a macrolide antibiotic. In some preferred embodiments, the antibiotic therapy comprises an oral regimen comprising doxycycline, amoxicillin, or cefuroxime axetil. In other embodiments, the antibiotic therapy comprises a parenteral regimen comprising ceftriaxone, cefotaxime, or penicillin G. For instance, in embodiments in which the subject is an outpatient, the antibiotic therapy comprises an effective amount of doxycycline if the subject is an outpatient. Alternatively, in embodiments in which the subject is hospitalized, the antibiotic therapy comprises an effective amount of ceftriaxone.

Moreover, the present disclosure provides kits comprising: (a) a plurality of oligonucleotides which hybridize to a plurality of genes comprising at least 2, 3, 4, 5, 6, 7, 8, 9, 10, 11, 12, 13, 14, 15, 16, 17, 18, 19 or all 20 genes of the group consisting of ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276; and (b) instructions for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores. The kits of the present disclosure are suitable for and may be used in conjunction with the methods of the preceding paragraphs.

### BRIEF DESCRIPTION OF THE DRAWINGS

FIG. 1 shows a flowchart of the gene expression sequencing method used to narrow down a list of significant genes from the whole transcriptome of two cohorts, as well as targeted RNA resequencing of four sample sets. Abbreviations: BC (British Columbia); CA (California); DEGs (differentially expressed genes); KNNXV (k-nearest neighbor cross validation); MD (Maryland); and TREx (targeted RNA expression resequencing).

FIG. 2 shows a flowchart of the machine learning method and sample sets used to define the Lyme disease gene expression classifier panel.

FIG. 3 shows a comparison of the accuracy and kappa statistics of ten different machine learning (ML) methods on the 10× cross validation of a training set of 30 Lyme samples and 65 control samples. The abbreviations used for the machine learning methods are as follows: glmnet=generalized linear models (Friedman et al., J Stat Softw, 33:1-22, 2010), svmr=radial support vector machine (Suykens and Vandewalle, Neural Process Lett, 9:2930399, 1999), svml=linear support vector machine (Suykens and Vandewalle, supra, 1999), rf=random forest (Breiman, Mach Learn, 45:5-32, 2001), nb=naïve bayes (Rohl et al., Comput Stat, 17:29-46, 2002), nnet=neural networks (Ripley, Pattern Recognition and Neural Networks, Cambridge University Press, 1996), pam=nearest shrunken centroids (Tibshirani et al., Proc Natl Acad Sci USA, 99:6567-6572, 2002), cart=classification and regression trees (Breiman et al., Classification and Regression Trees, Taylor & Francis, 1984), knn=k-nearest neighbor (Altman, Am Stat, 46:175-185, 1992), lda=linear discriminant analysis (Ripley, supra, 1996).

FIG. 4A-FIG. 4F show results from the Lyme disease gene expression classifier composed of 20 genes as defined by the generalized linear model machine learning algorithm. In this figure and associated experimental example, the disease score shown is a scaled Lyme score derived by scaling the raw Lyme score from 0.0 to 1.0 by the software package in R (see R-project website). The scaling was done for ease of visual representation with positive scores scaled to a value in a range greater than 0.5 and less than 1.0 (between 0.5 and 1.0=Lyme), and negative scores scaled to a value in a range greater than 0.0 and less than 0.5 (between 0.0 and 0.5=non-Lyme. FIG. 4A shows a chart of misclassification error depending on the number of genes considered (upper x-axis) and related log (lambda) statistic (lower x-axis). FIG. 4B shows a boxplot of the Lyme score for Lyme samples and control samples in the training set. FIG. 4C shows a receiver-operating-characteristic (ROC) curve of the performance of the Lyme classifier on a training set of 30 Lyme seropositive samples and 65 control samples. FIG. 4D shows a boxplot of the Lyme score for Lyme samples and control samples in the validation set. FIG. 4E shows a ROC curve of the performance of the Lyme classifier on a validation set of 30 Lyme seropositive samples and 65 control samples. FIG. 4F shows a boxplot of the Lyme score of validation samples from patients diagnosed with an EM rash separated by serological status: (1) Lyme seropositive; (2) late seroconverter (seroconverted during or after treatment); and (3) Lyme seronegative.

FIG. 5 shows a flowchart of an exemplary method for determining whether a subject has or does not have Lyme disease. The Lyme disease score is the sum of the gene expression scores (read counts) for each of the genes of the Lyme classifier multiplied by their respective gene weights plus an intercept value.

### DETAILED DESCRIPTION

Diagnosis of Lyme disease is often unreliable as it is typically made on the basis of tick exposure history and non-specific clinical findings. Erythema migrans, the “bull's-eye” rash associated with early Lyme disease, is seen less than 70% of patients and can be mistaken for other skin conditions and other diseases. For example, Southern tick associated rash illness (START), is also associated with the development of an erythematous bull's-eye rash around the tick bite, but is not caused by the Lyme agent (Borrelia burgdorferi in the United States) (Goddard, Am J Med, 130:231-233, 2017). Culture is impractical and rarely available, while serologic and nucleic acid testing for Borrelia have been of limited use due to low sensitivity. Moreover, Lyme disease serology often misses the window of early acute infection as patients present to the clinic prior to appearance of a detectable antibody response (Steere et al., Clin Infect Dis, 47:188-195, 2008).

Recent development of “omics” methods allow for the evaluation of novel diagnostic methods. The use of transcriptome profiling by next-generation sequencing (RNA-seq) is a promising approach to identify diagnostic host biomarkers in response to infection, such as tuberculosis (Anderson et al., N Eng J Med, 370:1712-1723, 2014), S. aureus bacteremia (Ahn et al., PLoS One, 8:e48979, 2013), or influenza (Woods et al., PLoS One, 8:e52198, 2013; and Zaas et al., Cell Host Microbe, 6:207-217, 2009). In the present disclosure, whole transcriptome sequencing and targeted RNA resequencing were used in conjunction with machine learning methods to define a panel of 20 human genes whose expression can distinguish samples from acute Lyme disease patients from controls.

The Lyme disease gene expression classifier provided in Table 1-5 showed a 94.4% sensitivity for detecting serologically positive Lyme samples in the validation set, and a 90% sensitivity for samples from Lyme disease patients that were seronegative at the time of sampling, but who seroconverted at a later stage. These results are much higher that the 29%-40% sensitivity reported for the detection of early Lyme disease infection (Steere et al., Clin Infect Dis, 47:188-195, 2008). Moreover, 16 out of 30 (53.3%) samples from patients clinically diagnosed with Lyme disease but who were consistently seronegative, were classified as Lyme using the methods of the present disclosure. As such, the methods of the present disclosure allow for more accurate management of Lyme disease in patients with ambiguous laboratory results. Given that all Lyme patients included in this study had an EM rash≥5 cm and concurrent “flu-like” symptoms such as fever, and were enrolled from a region highly endemic for Lyme disease, it is likely that most serologically negative patients in this study were indeed infected with Borrelia, but it is not possible to ascertain that all were. It is thus possible that the Lyme gene expression classifier developed based on serologically positive patients might underestimate the true prevalence of Borrelia infection. In the absence of a gold standard diagnostic test, an approach using more than one method could help determine the presence of Lyme disease even more accurately.

A recent assay developed using metabolomics achieved 88% sensitivity of Lyme seropositive samples and 95% specificity on controls corresponding to healthy subjects from endemic and non-endemic areas, plus patients diagnosed with syphilis, severe periodontitis, infectious mononucleosis, or fibromyalgia (Molins et al., Clin Infect Dis, 60:1767-1775, 2015). The methods of the present disclosure fared better, albeit tested on a smaller number of samples (220 samples compared to 461 samples). Thus, the Lyme disease gene classifier panel (ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276) of the present disclosure is an important new tool for diagnosis of acute infection with Borrelia burgdorferi, especially during the early stages of infection, when IgM are not yet detectable, or in cases of seronegative Lyme disease (Rebman et al., Clin Rheumatol, 34:585-589, 2015; and Dattwyler et al., N Engl J Med, 319:1441-1446, 1988).

### I. Definitions

As used herein and in the appended claims, the singular forms “a,” “an” and “the” include plural referents unless otherwise indicated or clear from context. For example, “a polynucleotide” includes one or more polynucleotides.

It is understood that aspects and embodiments described herein as “comprising” include “consisting of” and “consisting essentially of” embodiments.

Reference to “about” a value or parameter describes variations of that value or parameter. For example, the term about when used in reference to 20% of ticks being suspected of being infected encompasses 18% to 22% of ticks being suspected of being infected.

The term “plurality” as used herein in reference to an object refers to three or more objects. For instance, “a plurality of genes” refers to three or more genes, preferably 3, 4, 5, 6, 7, 8, 9, 10, 11, 12, 13, 14, 15, 16, 17, 18, 19, 20, 25, 30, 35, 40, 45, or 50 more genes.

The term “portion” as used herein in reference to sequencing a member of an RNA expression library (e.g., mRNA or cDNA library) refers to determining the sequence of at least about 25, 50, 75, 100, 125, 150, 175, 200, 225, or 250 bases of the library member. In some embodiments, sequencing a portion may include sequencing the entire library member.

As used herein, the term “isolated” refers to an object (e.g., PBMC) that is removed from its natural environment (e.g., separated). “Isolated” objects are at least 50% free, preferably 75% free, more preferably at least 90% free, and most preferably at least 95% (e.g., 95%, 96%, 97%, 98%, or 99%) free from other components with which they are naturally associated.

As used herein, “a subject suspected of having a tick-borne disease” is a subject that meets one or more of the following criteria: has been bitten by a tick; has an erythema migrans rash; has flu-like symptoms (e.g., fatigue, fever, joint pain, and/or headaches); and has visited or resided in a region in which ticks are likely to be infected with a human pathogen (e.g., a bacterial, viral, or protozoal organism which is known to cause disease in infected humans).

The terms “treating” or “treatment” of a disease refer to executing a protocol, which may include administering one or more pharmaceutical compositions to an individual (human or other mammal), in an effort to alleviate signs or symptoms of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

### II. Methods for Measuring Gene Expression & Diagnosis of Acute Lyme Disease

Certain aspects of the present disclosure relate to methods for measuring gene expression, which may be used to assist in diagnosis of acute Lyme disease. In some embodiments, the methods include one or more techniques selected from of the group consisting of sequence analysis, hybridization, and amplification. For example, in some embodiments, the methods may include, without limitation, RT-qPCR, Luminex, Nanostring, and/or microarray. Exemplary methods are set forth below, but the skilled artisan will appreciate that various methods for measurement of gene expression that are known in the art can be employed without departing from the scope of the present disclosure.

In some embodiments, a method for measuring gene expression includes: (a) measuring RNA expression of a plurality of genes of peripheral blood mononuclear cells (PBMCs) isolated from a blood sample obtained from a mammalian subject suspected of having a tick-borne disease; (b) calculating a weighted RNA expression score for each of the plurality of genes; and (c) calculating a Lyme disease score by taking the sum of the weighted RNA expression scores. Thus, the gene expression of the plurality of genes forms the basis of the Lyme disease score used to diagnose acute Lyme disease. In some embodiments, the mammalian subject is a human. For example, in some embodiments, the Lyme disease score is the sum of the gene expression scores (read counts) for each of the genes of the Lyme classifier (plurality of genes) multiplied by their respective gene weights plus an intercept value (see Table 1-5). In some embodiments, the method further includes: step (d) identifying the subject as not having acute Lyme disease when the Lyme disease score is negative. In other embodiments, the method further includes: step (d) identifying the subject as having acute Lyme disease when the Lyme disease score is positive.

In some embodiments, the method further includes: obtaining a blood sample from the subject and isolating the PBMCs from the blood sample prior to step (a). The blood sample may be drawn into a container such as a cell preparation tube (CPT). For example, in some embodiments, the container used to collect the whole blood sample may include without limitation a BD Vacutainer® CPT™ Sodium Heparin or a BD Vacutainer® CPT™ EDTA. Subsequent to collection, PBMCs are isolated from the whole blood sample using a suitable cell separation method such as centrifugation through a polysaccharide density gradient medium (e.g., Ficoll-Paque® marketed by GE Healthcare, Lymphoprep® marketed by Alere Technologies AS, etc.).

In some embodiments, the method further includes: extracting RNA from the PBMCs prior to step (a). For example, in some embodiments, the method used to extract RNA may include, without limitation, Zymo Direct-zol™, TRIzol® (reagents for isolating biological material marketed by Molecular Research Center, Inc.), phenol/chloroform, etc. RNA extraction may also include treating the RNA with DNAse to remove DNA contamination, which may occur during the extraction process (e.g., in an RNA extraction kit including an on-column DNAse step) or after the extraction process (e.g., DNAse treatment of extracted RNA). Subsequent to extraction, RNA concentration may be measured using a method such as Qubit fluorometric quantitation.

In some embodiments, the plurality of genes used in the method includes at least 3, 4, 5, 6, 7, 8, 9, 10, 11, 12, 13, 14, 15, 16, 17, 18, 19, 20, 21, 22, 23, 24, 25, 50, 75, 100, 125, 150, or all 172 genes of the first gene panel of Table 1-4. In a subset of these embodiments, the plurality of genes used in the method includes at least 3, 4, 5, 6, 7, 8, 9, 10, 11, 12, 13, 14, 15, 16, 17, 18, 19, 20, 21, 22, 23, 24, 25, 50, 75, or all 86 genes of the second gene panel of Table 1-4. In some preferred embodiments, the plurality of genes used in the method includes at least 3, 4, 5, 6, 7, 8, 9, 10, 11, 12, 13, 14, 15, 16, 17, 18, 19, or all 20 genes of the group containing ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276 (third gene panel of Table 1-4). In some embodiments, the plurality of genes includes NCF1. In some embodiments, the plurality of genes includes ANXA5. In some embodiments, the plurality of genes includes CR1. In some embodiments, the plurality of genes includes STAB1. In some embodiments, the plurality of genes includes MLF11P.

A. Next Generation Sequencing Methods

In sequencing by synthesis, single-stranded DNA is sequenced using DNA polymerase to create a complementary second strand one base at a time. Most next generation (high-throughput) sequencing methods use a sequencing by synthesis approach, which is often combined with optical detection. High-throughput methods are advantageous in that many thousand (e.g., 10⁶-10⁹) sequences may be determined in parallel. Various high-throughput sequencing methods that may be used to measure gene expression in connection with the present disclosure are briefly described below.

Illumina (Solexa) sequencing, is a high-throughput method that uses reversible terminator bases for sequencing by synthesis (see e.g., Bentley et al., Nature, 456:53-59, 2008; and Meyer and Kircher, “Illumina Sequencing Library Preparation for Highly Multiplexed Target Capture and Sequencing”. Cold Springs Harbor Protocols 2010: doi:10.1101/pdb.prot5448). First, DNA molecules are attached to a slide and amplified to generate local clusters of the same DNA sequence. Then, four types of fluorescently labeled nucleotides with reversible 3′ blockers (reversible terminator bases or RT-bases) are added to the chip, the excess is washed away, and the chip is imaged. After imaging, the dye and the 3′ blocker are removed from the nucleotide, and the next round of RT-bases is added to the chip and imaged.

Pyrosequencing is another type of sequencing by synthesis method that detects the release of pyrophosphate (PPi) during DNA synthesis (see, e.g., Ronaghi et al., Science, 281:363-365, 1998). In order to detect PPi, ATP sulfurylase, firefly luciferase, and luciferin are used, which together act to generate a visible light signal from PPi. Light is produced when a nucleotide has been incorporated into the complementary strand of DNA by DNA polymerase, and the intensity of the light emitted is used to determine how many nucleotides have been incorporated. Each of the four nucleotides is added in turn until the sequence is complete. High-throughput pyrosequencing, also known as 454 pyrosequencing (Roche Diagnostics), uses an initial step of emulsion PCR to generate oil droplets containing a cluster of single DNA sequences attached to a bead via primers. These droplets are then added to a plate with picoliter-volume wells such that each well contains a single bead as well as the enzymes needed for pyrosequencing.

Ion semiconductor sequencing (Ion Torrent, now Life Technologies) is a further type of sequencing by synthesis method that uses the hydrogen ions released during DNA polymerization for sequencing (see, e.g., U.S. Pat. No. 7,948,015). First, a single strand of template DNA is placed into a microwell. Then, the microwell is flooded with one type of nucleotide. If the nucleotide is complementary, it is incorporated into the secondary strand, and a hydrogen ion is released. The release of the hydrogen ion triggers a hypersensitive ion sensor; if multiple nucleotides are incorporated, multiple hydrogen ions are released, and the resulting electronic signal is higher.

Sequencing by ligation (SOLiD sequencing marketed by Applied Biosystems) uses the mismatch sensitivity of DNA ligase in combination with a pool of fluorescently labeled oligonucleotides (probes) for sequencing (see, e.g., WO 2006084132). First, DNA molecules are amplified using emulsion PCR, which results in individual oil droplets containing one bead and a cluster of the same DNA sequence. Then, the beads are deposited on a glass slide. The probes are added to the slide along with a universal sequencing primer. If the probe is complementary, the DNA ligase joins it to the primer, fluorescence is measured, and then the fluorescent label is cleaved off. This leaves the 5′ end of the probe available for the next round of ligation.

Third-generation or long-read sequencing methods are high-throughput sequencing methods that sequence single molecules. These methods do not require initial PCR amplification steps. Single-molecule real-time sequencing (Pacific Biosciences) is a sequencing by synthesis long-read sequencing method, which employs zero-mode waveguides (ZMWs), which are small wells with capturing tools located at the bottom (see, e.g., Levene, Science, 299:682-686, 2003; and Eid et al., Science, 323:133-138, 2009). In brief, one DNA polymerase enzyme is attached to the bottom of a ZMW, and a single molecule of single-stranded DNA is present as a template. Four types of fluorescently-labelled nucleotides are present in a solution added to the ZMWs. When a nucleotide is incorporated into the second strand by the DNA polymerase in a ZMW, the fluorescence is detected by the capturing tools at the bottom of the ZMW. Then, the fluorescent label is cleaved off and diffuses away from the capturing tools at the bottom of the ZMW so it is no longer detectable and the remaining DNA strand in the ZMW is free of labels.

Nanopore sequencing (Oxford nanopore) is a sequencing method that sequences a single DNA or RNA molecule without any form of label. The principle of nanopore sequencing is that DNA passing through a nanopore changes the ion current of the nanopore in a manner dependent on the type of nucleotide. The nanopore itself contains a detection region able to recognize different nucleotides. Current nanopore sequencing methods in development are either solid state methods employing metal or metal alloys (see, e.g., Soni et al., Rev Sci Instrum, 81(1): 014301, 2010) or biological employing proteins (see, e.g., Stoddart et. al.., Proc Natl Acad Sci USA, 106:7702-7707, 2009).

Further large-scale sequencing techniques for use in measuring gene expression in connection with methods of the present disclosure include but are not limited to microscopy-based techniques (e.g., using atomic force microscopy or transmission electron microscopy), tunneling currents DNA sequencing, sequencing by hybridization (e.g., using microarrays), sequencing with mass spectrometry (e.g., using matrix-assisted laser desorption ionization time-of-flight mass spectrometry, or MALDI-TOF MS), microfluidic Sanger sequencing, RNA polymerase (RNAP) sequencing (e.g., using polystyrene beads), and in vitro virus high-throughput sequencing.

Serial analysis of gene expression (SAGE) is a method that allows quantitative measurement of gene expression profiles that can be compared between samples (Velculescu et al., Science, 270: 484-7, 1995). First, cDNA is synthesized from an RNA sample. Then, through multiple steps involving bead binding, cleavage, and adapters, short cDNA fragments (tags) are produced. These tags are concatenated, amplified using bacteria, isolated, and finally sequenced using high-throughput sequencing techniques. SAGE can be used to measure gene expression changes of multiple genes at once, for example in response to infection.

Specifically, in some embodiments of the present disclosure, measuring RNA expression of a plurality of genes includes targeted RNA expression resequencing including: (i) preparing an RNA expression library for the plurality of targeted genes from RNA extracted from the PBMCs; (ii) sequencing a portion of at least 50,000 members of the library; and (iii) generating a read count for RNA expression of the plurality of genes by normalization to the sequence of the at least 50,000 members of step (ii). In other embodiments, measuring RNA expression of a plurality of genes includes whole transcriptome shotgun sequencing (WTSS) including: (i) preparing an RNA expression library for the plurality of genes from RNA extracted from the PBMCs; (ii) sequencing a portion of at least 1,000,000 members of the library; and (iii) generating a read count for RNA expression of the plurality of genes by normalization to the sequence of the at least 1,000,000 members of step (ii). For example, library preparation may include, without limitation, the use of the Illumina TruSeq targeted RNA expression kit. The sequencing done in step (ii) of the above two embodiments may be, without limitation, Illumina MiSeq single-end reads 50 base pairs in length with a target sequencing depth of 200,000 reads per sample. The read count in step (iii) may be generated using any RNA library sequencing analysis methods (e.g., pipelines) known in the art. For example, these methods may include, without limitation, TopHat-Cufflinks, MiSeq reporter targeted RNA workflow, R software packages, graph-based analysis packages, and/or a combination thereof. In some embodiments, step (b) includes multiplying the read count for each of the plurality of genes by a predetermined gene expression weight to obtain the weighted RNA expression score (see Table 1-5). For example, in some embodiments, the predetermined gene expression weight may be calculated by an algorithm using additional information about the subject selected from the group containing age, sex, symptoms, time elapsed since tick bite, and/or previous Lyme disease diagnosis.

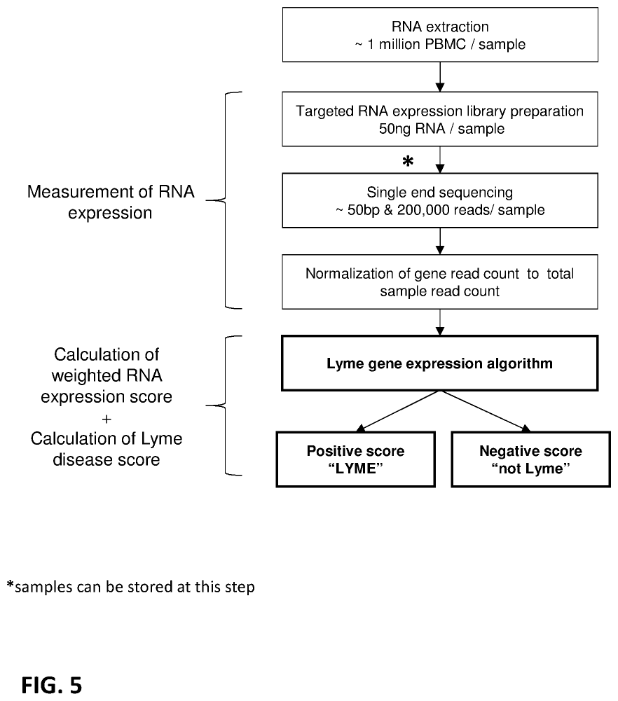

An exemplary method of measuring gene expression and diagnosing acute Lyme disease is illustrated in FIG. 5. As shown in FIG. 5, the process starts with RNA extraction from a sample containing about 1 million PBMCs. In the second step of the process, a targeted RNA expression library is prepared from a sample containing 50 ng of RNA. The expression library is targeted to a plurality of genes, as described above. After this second step, the samples can be stored for later processing. In the third step, the prepared library is sequenced using single end sequencing of about 50 base pairs, and a sequencing depth of 200,000 reads per sample. After the library is sequenced, the gene read count is normalized to the total sample read count in the fourth step. At the end of step four, the portion of the method used for RNA expression measurement (i.e. gene expression measurement) is complete. The fifth step is the first part of the portion of the method used for diagnosing acute Lyme disease. A Lyme gene expression algorithm is used to calculate the weighted RNA expression score. As described above, this Lyme gene expression algorithm may include additional information about the subject. In step six, the Lyme disease score is then calculated by taking the sum of the weighted RNA expression score. If the Lyme disease score is positive, the subject is diagnosed with Lyme disease, whereas if the Lyme disease score is negative, the subject is not diagnosed with Lyme disease.

B. Amplification Methods for Measuring Gene Expression

Methods that may be used to measure gene expression in connection with the present disclosure may include an amplification step. In some embodiments of the present disclosure, measuring RNA expression of a plurality of genes includes a quantitative polymerase chain reaction (qPCR). For instance, some methods include performing reverse transcriptase-quantitative polymerase chain reaction (RT-qPCR) on RNA extracted from the PBMCs. Quantitative reverse transcription polymerase chain reaction (qRT-PCR) is an amplification method that uses fluorescence to quantitatively measure gene expression (see, e.g., Heid et al., Genome Res 6:986-994, 1996). The first step of qRT-PCR is to produce complementary DNA (cDNA) by reverse transcribing mRNA. The cDNA is used as the template in the PCR reaction. In addition to the template, gene-specific primers, a buffer (and other reagents for stability), a DNA polymerase, nucleotides, and a fluorophore are added to the PCR reaction. The reaction is then placed in a thermocycler that is able to both cycle through the different temperatures required for the standard PCR steps (e.g., separating the two strands of DNA, primer binding, and DNA polymerization) and illuminate the reaction with light at a particular wavelength to excite the fluorophore. Over the course of the reaction, the level of fluorescence is detected, and this level is subsequently used to quantify the amount of gene expression.

The use of fluorescence in qRT-PCR can be done in two different ways. The first way uses a dye in the reaction mixture that fluoresces when it binds to double stranded DNA. The intensity of the fluorescence increases as the amount of double stranded DNA increases, but the dye is not specific for a particular sequence. The second way uses sequence-specific probes labeled with a fluorescent reporter. The intensity of the fluorescence increases as the amount of the particular sequence increases.

C. Hybridization Methods for Measuring Gene Expression

Methods that may be used to measure gene expression in connection with the present disclosure may include a hybridization step. In some preferred embodiments, the methods include use of a DNA microarray. DNA microarrays employ a plurality of specific DNA sequences (e.g., probes, reporters, oligos) attached to a slide or chip. First, cDNA from a sample is labeled with a fluorophore, silver, or a chemiluminescent molecule. Then, the labeled sample is hybridized to the DNA microarray under specific conditions, and hybridization is subsequently detected and quantified. Other methods of measuring gene expression through hybridization include but are not limited to Northern blot analysis, and in situ hybridization.

### III. Methods for Treating Lyme Disease

Certain aspects of the present disclosure relate to methods for treating Lyme disease. Exemplary methods of treatment are set forth below. Any of the methods for measuring gene expression described herein can be used for diagnosis or confirmation of acute Lyme disease in a subject in conjunction with treating Lyme disease. In some embodiments, treating Lyme disease includes administering an antibiotic therapy to the subject to treat the Lyme disease. In some embodiments, the antibiotic therapy includes an effective amount of an antibiotic selected from the group including: tetracyclines, penicillins, and cephalosporins. In other embodiments, the antibiotic therapy includes an effective amount of macrolides. In some embodiments, the antibiotic therapy includes an oral regimen including doxycycline, amoxicillin or cefuroxime axetil. In other embodiments, the antibiotic therapy includes a parenteral regimen including doxycycline, amoxicillin or cefuroxime axetil. In some embodiments, the antibiotic therapy includes an effective amount of doxycycline if the subject is an outpatient. In other embodiments, the antibiotic therapy includes an effective amount of ceftriaxone if the subject is hospitalized.

### IV. Kits for Measuring Gene Expression & Diagnosis of Acute Lyme Disease

Certain aspects of the present disclosure relate to kits for measuring gene expression and diagnosis of acute Lyme disease. In some embodiments, the kit includes: (a) a plurality of oligonucleotides which hybridize to a plurality of genes; and (b) instructions for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores. In some embodiments, the plurality of genes used includes at least 2, 3, 4, 5, 6, 7, 8, 9, 10, 11, 12, 13, 14, 15, 16, 17, 18, 19, 20, 21, 22, 23, 24, 25, 50, 75, 100, 125, 150, or all 172 genes of the first gene panel of Table 1-4. In a subset of these embodiments, the plurality of genes includes at least 2, 3, 4, 5, 6, 7, 8, 9, 10, 11, 12, 13, 14, 15, 16, 17, 18, 19, 20, 21, 22, 23, 24, 25, 50, 75, or all 86 genes of the second gene panel of Table 1-4. In some embodiments, the plurality of genes comprises at least 2, 3, 4, 5, 6, 7, 8, 9, 10, 11, 12, 13, 14, 15, 16, 17, 18, 19 or all 20 genes of the group consisting of ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276. In some embodiments, the plurality of oligonucleotides of the kit are attached to a slide or a chip. In some embodiments, the plurality of oligonucleotides of the kit each comprise a label for ease in detection. In some embodiments, the plurality of oligonucleotides comprise a pair of oligonucleotides for each of the plurality of genes. In some embodiments, the sequence of the pair of oligonucleotides is set forth in Table 1-1.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

In the experimental disclosure which follows, the following abbreviations apply: AUC (area under the curve); CART (classification and regression trees); DEG (differentially expressed gene); EM (erythema migrans); FPKM (fragments per kilobase of exon per million fragments mapped); GLMNET (generalized linear models); KNN (k-nearest neighbor); KNNXV (k-nearest neighbor cross validation); LDA (linear discriminant analysis); NB (naïve bayes); NGS (next-generation sequencing); NNET (neural networks); PAM (nearest shrunken centroids); PBMCs (peripheral blood mononuclear cells); RF (random forest); RPART (classification and regression trees); ROC (receiver-operating-characteristic curves); SVML (linear support vector machine); SVMR (radial support vector machine); and TREx (targeted RNA expression resquencing).

### Example 1

### Gene Expression Classifier for the Early Detection of Lyme Disease

### Materials and Methods

The participants enrolled in this study were 90 Lyme disease patients and 26 matched control patients from Baltimore, Md., which is an area highly endemic for Lyme disease. All 90 Lyme disease participants included in this study presented with a physician documented erythema migrans (EM) of ≥5 cm and concurrent flu-like symptoms that included at least one of the following; fever, chills, fatigue, headache and new muscle or joint pains. Two-tier serological Lyme disease testing was performed on EM patients at the first visit and following completion of the standard 3-week course of doxycycline treatment. All of the 26 matched control patients were required to have a negative Lyme test in order to be enrolled in the study.

In addition to the control participants, further control samples were also included in the study. A total of 82 additional control samples were collected in San Francisco, Calif., of which 37 were from healthy blood donors, 30 were from patients with flu, and 15 were from patients with bacteremia. An additional 20 control samples were collected in Vancouver, British Columbia, Canada, of which 10 were from tuberculosis patients, and 10 were from matched control patients. Patients in these two locations were diagnosed with flu, bacteremia or tuberculosis based on expert clinical observation, chart review and positive diagnostic test by NxTag Respiratory Pathogen Panel (Luminex Corp., Austin, Tex.), standard bacterial culture, and T-SPOT.TB blood test for tuberculosis (Oxford Diagnostic Laboratories, Marlborough, Mass.), respectively. Two-tier Lyme disease serology was not performed at the time of sampling, but was likely negative based on symptoms, clinical history and low Lyme endemicity in these areas.

Each of the samples began as a fresh whole blood sample, and then PBMCs were isolated from the samples using Ficoll® (Ficoll-Paque Plus, GE Healthcare). After isolating PBMCs, total RNA was extracted from 10⁷ PBMCs using TRIzol reagent (Life Technologies). Messenger RNA (mRNA) was isolated from the total RNA using the Oligotex mRNA mini kit (Qiagen). The isolated mRNA was used to generate RNA-Seq libraries using the Scriptseq RNA-Seq library preparation kit (Epicentre) according to the manufacturer's protocol. The RNA-Seq libraries were then sequenced on a Hiseq 2000 instrument (Illumina).

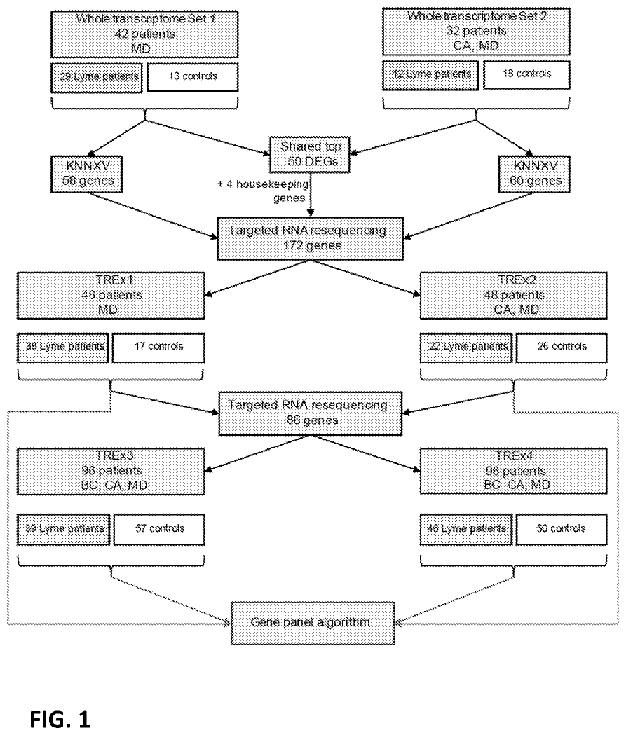

The samples were processed in two sets (FIG. 1). Set 1 corresponded to samples from 29 Lyme disease patients and 13 matched control patients (Bouquet et al., mBio 7, e00100-116, 2016). Set 2 corresponded to samples from 6 new Lyme disease patients and 6 matched control patients that were prepared and sequenced alongside samples from 6 flu patients and 6 bacteremia patients.

Data analysis of the RNA-Seq library sequencing described above began by mapping the paired-end reads to the human genome (February 2009 human reference sequence [GRCh37/hg19] produced by the Genome Reference Consortium). After mapping, the exons were annotated and FPKM (fragments per kilobase of exon per million fragments mapped) values for all 25,278 expressed genes were calculated using version 2 of the TopHat-Cufflinks pipeline (Kim et al., Genome Biol, 14:R36, 2013). The differential expression of genes was calculated by using the ‘variance modeling at the observational level’ (voom) transformation (Law et al., Genome Biol, 15:R29, 2014), which applies precision weights to the matrix count, followed by linear modeling with the Limma package (Ritchie et al., Nucleic Acids Res, 43:e47, 2015). Genes were considered to be differentially expressed when the change was greater than 1.5-fold, the P value was 0.05, and the adjusted P value (or false discovery rate) was 0.1% (Dalman et al., BMC Bioinformatics, 13Suppl2:S11, 2012).

After the whole transcriptome analysis, a custom panel of transcripts of interest was selected for targeted RNA resequencing. The quantitative analysis of this custom panel was performed using a targeted RNA enrichment resequencing approach that used anchored multiplex PCR, and was done on a large number of samples. Here, PBMC samples (˜1 million cells) were extracted using Zymo Direct-zol™ RNA miniprep with on-column DNase following the manufacturer's instructions. Reverse transcription was performed on 50 ng of RNA following the manufacturer's instructions from the Illumina TruSeq targeted RNA expression kit. Briefly, a custom panel of oligonucleotides (oligos), each capable of specifically hybridizing to one of the genes of interest, was designed and ordered using the Illumina DesignStudio platform. The oligos to genes of an exemplary 20 gene Lyme disease classifier panel are shown in in Table 1-1. This pool of oligos attached to a small RNA sequencing primer (smRNA) binding site was used to hybridize, extend and ligate the second strand of cDNA from our genes of interest. Amplification was then performed using primers with a complementary smRNA sequence, multiplexing index sequences, and sequencing adapters. The resulting libraries were sequenced on an Illumina Miseq to a depth of 2,500 reads/sample/gene. Gene expression count/sample/gene was performed on the instrument by Miseq reporter targeted RNA workflow (revision C). Briefly, following demultiplexing and fastq file generation, reads from each samples were aligned locally against references corresponding to targeted regions of interest using a banded Smith-Waterman algorithm (Okada et al., BMC Bioinformatics, 16:321, 2015). Normalization against the total number of reads from each sample and the machine learning algorithm were both done using R (see R-project website).

TABLE 1-1
Lyme Disease Classifier Oligonucleotides

| Gene symbol | Upstream Locus Specific Oligo | Downstream Locus Specific Oligo |
|---|---|---|
| ANXA5 | AGAATTTTGCCACCTCTCTTTATTCCA | GACTATAAGAAAGCTCTTCTGCTGCTC |
| | (SEQ ID NO: 1) ANXA5 | (SEQ ID NO: 2) ANXA5-rv |
| C3orf14 | CCACTTCCACGGCCTGAGGTGGTTTCT | TTACTGGGCATCAGTAGAAGAATATATTCC |
| | (SEQ ID NO: 3) C3orf14-fw | (SEQ ID NO: 4) C3orf14-rv |
| CDCA2 | TCCATTCCGAGCATCCGAAGACT | CAGTTCAAATGGCAAACTGGAAGAAGTG |
| | (SEQ ID NO: 5) CDCA2-fw | (SEQ ID NO: 6) CDCA2-rv |
| CR1 | GTGGTGCTGCTTGCGCTGCCGGT | CAGAATGGCTTCCATTTGCCAGGCCTA |
| | (SEQ ID NO: 7) CR1-fw | (SEQ ID NO: 8) CR1-rv |
| GBP2 | ACCTTCTTTCCAGTGCTAAAGGATCTC | GAACAACACCCTGGACATGGCT |
| | (SEQ ID NO: 9) GBP2-fw | (SEQ ID NO: 10) GBP2-rv |
| IFI27 | TCAGCTTCACATTCTCAGGAACTCTC | TCTGGCTGAAGTTGAGGATCTCTTAC |
| | (SEQ ID NO: 11) IFI27-fw | (SEQ ID NO: 12) IFI27-rv |
| ITGAM | GCCATGGCTCTCAGAGTCCTTCTGTTAA | GTTCAACTTGGACACTGAAAACGCA |
| | (SEQ ID NO: 13) ITGAM-fw | (SEQ ID NO: 14) ITGAM-rv |
| KCNJ2 | ATGTCCCCATGCTCCTGCGCCAGCAA | ATGTTCTCTGGATGTCAGCTGAGTCA |
| | (SEQ ID NO: 15) KCNJ2-fw | (SEQ ID NO: 16) KCNJ2-rv |
| KIF4A | GGCCCAGGGAGAACGGGGAAGGGACATTTA | TGAGATAGGATCATGAAGGAAGAGGTG |
| | (SEQ ID NO: 17) KIF4A-fw | (SEQ ID NO: 18) KIF4A-rv |
| MLF1IP | ACTTTAGAAAGAACACATTCCATGAAAG | AAAGCTGGTCAAAAGTGCAAGCCT |
| | (SEQ ID NO: 19) MLF1IP-fw | (SEQ ID NO: 20) MLF1IP-rv |
| NCF1 | GGCCCAACGCCAGATCAAGCGG | TCGTCCATCCGCAACGCGCACAGCAT |
| | (SEQ ID NO: 21) NCF1-fw | (SEQ ID NO: 22) NCF1-rv |
| PLBD1 | CTAACCCAAGTCCTGGAGGTTGTTATG | TGGCAGATATCTACCTAGCATCTCAGT |
| | (SEQ ID NO: 23) PLBD1-fw | (SEQ ID NO: 24) PLBD1-rv |
| PLK1 | GCAGCGTGCAGATCAACTTCTTC | ACACCAAGCTCATCTTGTGCCCA |
| | (SEQ ID NO: 25) PLK1-fw | (SEQ ID NO: 26) PLK1-rv |
| RAD51 | CTTTATCAAGCATCAGCCATGATGGTAG | TGCACTGCTTATTGTAGACAGTGCCA |
| | (SEQ ID NO: 27) RAD51-fw | (SEQ ID NO: 28) RAD51-rv |
| SLC25A37 | ACCCTGCTCCACGATGCGGTAATGAAT | TGCAGATGTACAACTCGCAGCA |
| | (SEQ ID NO: 29) SLC25A37-fw | (SEQ ID NO: 30) SLC25A37-rv |
| STAB1 | TGGCAGGCTTCAGCTTCGTCAG | GCTGTGATGTGAAAACCACGTTTGTC |
| | (SEQ ID NO: 31) STAB1-fw | (SEQ ID NO: 32) STAB1-rv |
| STEAP4 | GCAGTCAACTGGAGAGAGTTCCGATTT | GACCCTGATCTTGTGTACAGCCCA |
| | (SEQ ID NO: 33) STEAP4-fw | (SEQ ID NO: 34) STEAP4-rv |
| TBP | CTCCTTATTTTTGTTTCTGGAAAAGTTGT | CTAAAGTCAGAGCAGAAATTTATGAAGC |
| | (SEQ ID NO: 35) TBP-fw | (SEQ ID NO: 36) TBP-rv |
| TNFSF13B | TATTGGTCAAAGAAACTGGTTACTTTTT | TGATAAGACCTACGCCATGGGACAT |
| | (SEQ ID NO: 37) TNFSF13B-fw | (SEQ ID NO: 38) TNFSF13B-rv |
| ZNF276 | CGCTACCTGCAGCGCCACGTGAAGCTCAT | TGTGACGAATGTGGACAAACCTTCAAG |
| | (SEQ ID NO: 39) ZNF276-fw | (SEQ ID NO: 40) ZNF276-rv |

The k-nearest neighbor classification with leave-one-out cross validation algorithm (KNNXV) (Golub et al., Science, 286:531-537, 1999), as implemented on Genepattern (Reich et al., Nat Genet, 38:500-501, 2006), was used to classify the samples. This algorithm was used on each whole transcriptome differentially expressed genes set with a k of three, signal to noise ratio feature selection, Euclidean distance, and by iteratively decreasing the number of features until reaching maximum accuracy.

Class prediction accuracy on targeted RNA resequencing readcount results was tested using the caret package (Kuhn, J Stat Softw, 28:1-26, 2008) in R software, version 3.01 (R Project for Statistical Computing) for 10 different machine learning methods at default parameters: classification and regression trees (‘rpart’ method) (Breiman et al., Classification and Regression Trees, Taylor & Francis, 1984), generalized linear models (‘glmnet’ method) (Friedman et al., J Stat Softw, 33:1-22, 2010), linear discriminant analysis (‘lda’ method) (Ripley, Pattern Recognition and Neural Networks, Cambridge University Press, 1996), k-nearest neighbor (‘knn’ method) (Altman, Am Stat, 46:175-185, 1992), random forest (‘rf’method) (Breiman, Mach Learn, 45:5-32, 2001), naïve bayes (‘nb’ method) (Rohl et al., Comput Stat, 17:29-46, 2002), neural networks (‘nnet’ method) (Ripley, Pattern Recognition and Neural Networks, Cambridge University Press, 1996), linear and radial support vector machine (‘svmLinear’ and ‘svmRadial’ methods) (Suykens and Vandewalle, Neural Process Lett, 9:2930399, 1999), and nearest shrunken centroids (‘pam’ method) (Tibshirani et al., Proc Natl Acad Sci USA, 99:6567-6572, 2002). Subsequent computing of the generalized linear models were run with a lasso (least absolute shrinkage and selection operator) penalty.

The performance of the classifier (KNNXV) was evaluated with the use of receiver-operating-characteristic curves (ROC), calculation of area under the curve (AUC) (Hanley and McNeil, Radiobiology, 143:29-36, 1982), and estimates of sensitivity, specificity, negative predictive value, positive predictive value, and the negative likelihood ratio (defined as (1−sensitivity)÷specificity).

The Mann-Whitney nonparametric test was used for the analysis of continuous variables, and Fisher's exact test was used for categorical variables. All confidence intervals were reported as two-sided binomial 95% confidence intervals. Statistical analysis was performed with R software, version 3.01 (R Project for Statistical Computing).

### Results

No significant differences in age or sex were noted between the 90 Lyme disease patients and 26 matched control patients from Baltimore, Md. (Table 1-2). The two-tiered antibody test for Lyme was positive in 36 of 90 patients at the pre-treatment visit (40%), an additional 24 of 90 (26.7%) seroconverted during treatment, and 30 of 90 (33.3%) remained seronegative post-treatment. Similarly, no significant differences in age or sex were noted between the 37 healthy blood donors and the 45 patients with bloodstream infections from San Francisco, Calif. (Table 1-2). Of the 45 patients with bloodstream infections, 15 patients were diagnosed with bacteremia caused by Enterococcus faecium, Escherichia coli, Klebsiella pneumoniae, Staphylococcus aureus, Staphylococcus epidermidis, or Streptococcus pneumoniae as evidenced by standard plate culture, and 30 patients were diagnosed with Influenza A as evidenced by the Luminex NxTAG respiratory pathogen panel. Finally, no significant differences in age or sex were noted between the 10 tuberculosis patients and the 10 matched control patients from Vancouver, British Columbia, Canada (Table 1-2). The 10 patients with tuberculosis were diagnosed using T-SPOT.TB (Oxford Immunotec).

TABLE 1-2
Demographic Of Patients With Early Lyme Disease And Healthy Controls

| Disease Cohort | Age | | | | Positive Lyme | | P-value | P-value |
|---|---|---|---|---|---|---|---|---|
| & Location | (Avg/IQR/Range) | | Females | | Serology¹ | | Age² | Sex³ |
| Lyme disease | 51 (42-64) | [20-78] | 46/90 | (51.1%) | 60/90 | (66.7%) | 0.32 | 0.82 |
| MD, USA | | | | | | | | |
| Healthy 1 | 55 (45-65) | [22-73] | 15/28 | (53.6%) | 0/26 | (0%) | | |
| MD, USA | | | | | | | | |

| | | | | | | | |
|---|---|---|---|---|---|---|---|
| Tuberculosis | 54 (42-68) | [22-76] | 3/10 | (30%) | ND | 0.68 | 0.18 |
| BC, Canada | | | | | | | |
| Healthy 2 | 51 (39-65) | [36-71] | 6/10 | (60%) | ND | | |
| BC, Canada | | | | | | | |
| Flu | 59 (36-82) | [4-104] | 12/30 | (40%) | ND | 0.06 | 0.74 |
| CA, USA | | | | | | | |
| Bacteremia | 59 (53-69) | [23-81] | 7/15 | (46.7%) | ND | | |
| CA, USA | | | | | | | |
| Healthy 3 | 51 (46-59) | [31-71] | 17/37 | (45.9%) | ND | | |
| CA, USA | | | | | | | |

¹2-tier.
²Disease versus control age.
³Disease versus control sex.

As described in the previous section, the samples were divided into Set 1 and Set 2 and next generation sequencing using RNA-Seq was performed to quantify the global transcriptome response. Results from whole transcriptome Set 1 (FIG. 1) were as previously reported (Bouquet et al., mBio, 7:e00100-116, 2016. Briefly, an average of 82.5 (±48 s.d.) million raw reads for Set 1 and 30 (±17 s.d.) million raw reads for Set 2 were generated per sample. Sample Set1_Lyme29 was not included in the pooled analysis due to insufficient read counts. The batch effect was evaluated by principal component analysis over the expression values for all genes. Samples from Set 1 clustered separately from samples from Set 2. In order to remedy this batch effect, differential expression and KNNXV were calculated separately on each whole transcriptome set (FIG. 1). Iterative KNNXV found that a panel of 58 genes for Set 1 and a panel of 60 genes for Set 2 gave the best accuracy. These genes were combined with the top 50 differentially expressed genes shared between the two whole transcriptome datasets and four housekeeping genes to design a gene set for the targeted RNA resequencing assay (172 target genes total, listed in Table 1-3) used to test more samples (FIG. 1).

A maximum of 48 samples at a time could be sequenced on a single Illumina Miseq run, and tested with the assay targeting the expression of 172 genes as described above. Two sequencing runs (TREx1 and TREx2) and a total of 96 samples were tested using this assay (FIG. 1). The assay was then redesigned to target half of the genes included in the first panel in order to double the number of samples that could be multiplexed in a single sequencing run (86 target genes total, listed in Table 1-4). Welch's t-test was used to evaluate which 86 genes out of 172 showed the highest difference in expression value distribution between the Lyme and Control (consisting of samples from healthy, flu, and bacteremia patients) sample categories. Two sequencing runs (TREx3 and TREx4) tested these 86 genes on a total of 172 samples. Finally, all of the targeted RNA resequencing data (runs TREx1-TREx4) for those 86 genes was combined to test 10 different machine learning methods and devise the most accurate gene panel algorithm.

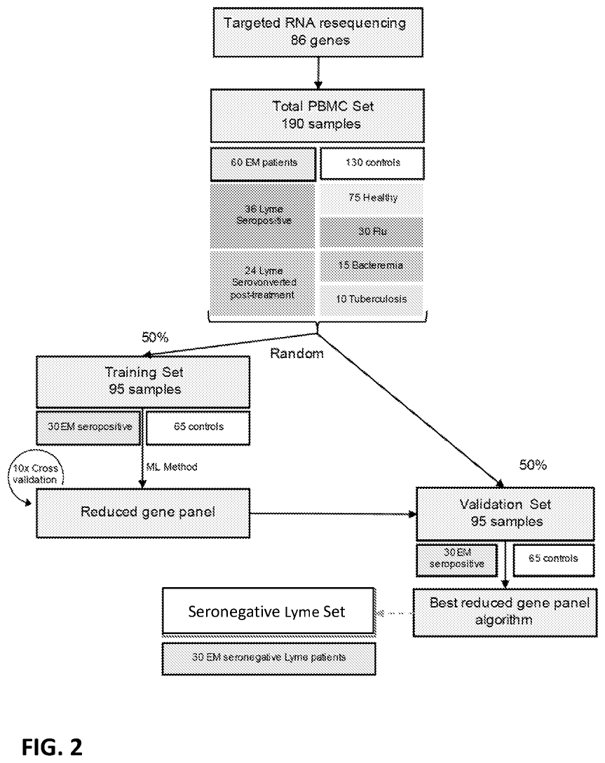

Machine learning methods were tested on targeted RNA resequencing data according to methods summarized in FIG. 2. Briefly, machine learning methods were trained and validated on a set of 190 unique samples. Lyme disease samples had to come from patients who were seropositive either at their first doctor visit or by the end of antibiotic treatment. Seronegative Lyme patients were not used to design the Lyme diagnostic panel, because of the risk of misdiagnosis based on symptomatology alone. Instead, the performance of the gene panel algorithm was first evaluated and defined using only samples from seropositive Lyme disease patients, and subsequently tested using samples from seronegative Lyme patients.

Seropositive Lyme samples and all control samples were randomly divided into a training set (50%) and a validation set (50%). Each machine learning method was evaluated on the training set using a 10× cross validation scheme.

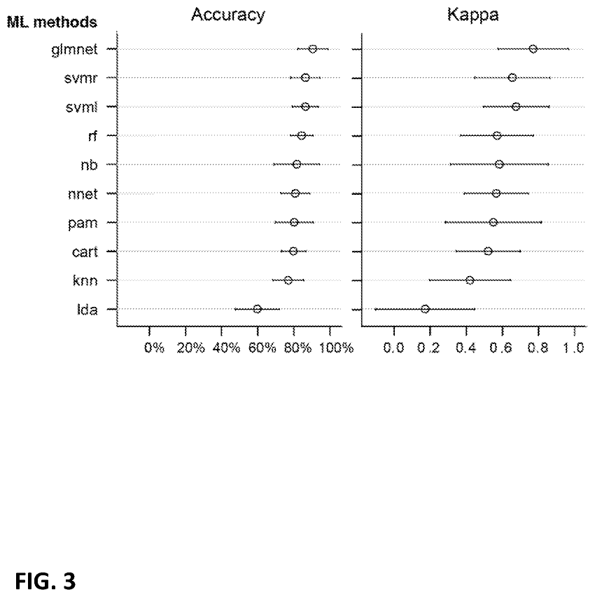

Generalized linear models as implemented by the glmnet package were found to provide the highest accuracy at 90.6% (IQR, 82.2%-100%) and kappa statistic at 0.77 (IQR, 0.57-1) (FIG. 3). The kappa statistic corresponds to the inter-rater agreement statistic for categorical items. Other methods, including support vector machine, random forest, naïve bayes, neural networks, nearest shrunken centroids, classification and regression trees, and k-nearest neighbor, also showed promising categorical discrimination accuracy on the training set (>79.9%), with the exception of the linear discriminant method which resulted in a 59.8% accuracy (FIG. 3).

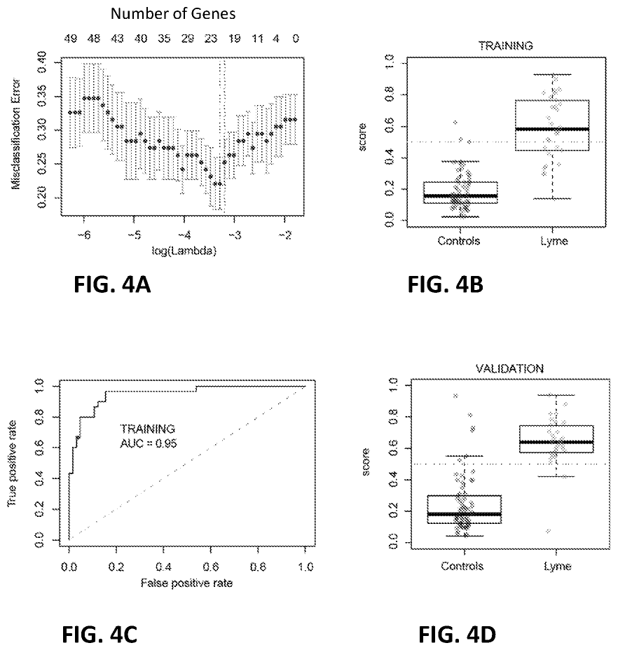

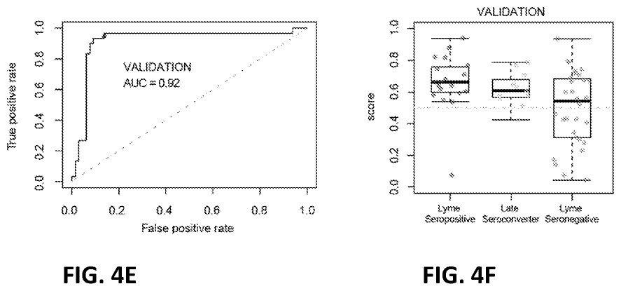

The generalized linear model method found that a panel of 20 genes (FIG. 4A, listed in Table 1-5) gave the lowest misclassification error on the training set (0.22 [0.18-0.26]). A disease score from 0.0 to 1.0 was calculated based on the expression of the 20 genes in the algorithm. A disease score greater than 0.5 classified the sample as Lyme and a score less than 0.5 classified the sample as a non-Lyme sample (healthy or other disease). The raw and scaled disease scores are shown in subsequent tables after rounding to the nearest 1×10⁻⁸ for readability. As such, indeterminate scaled disease scores of 0.50000000 are expected to be highly unlikely occurrences. Thus, a scaled disease score of 0.49998 would be indicative of Lyme disease and a scaled disease score of 0.5000003 would be indicative of no Lyme disease.

The intercept value (and gene weights) of Table 1-5 were based on measurement of expression of the specific 20 genes of interest using targeted RNA sequencing. For this reason, if expression of fewer or more than 20 genes is measured, then the intercept value and gene weights may differ somewhat from the exemplary values. Similarly, if gene expression was measured using a different method, then the intercept value and gene weights may differ somewhat from the exemplary values. Targeted RNA sequencing results in infinite values expressed as read counts, which are dependent on the total sequencing depth. qRT-PCR on the other hand, results in finite values expressed in Ct (cycle threshold) in a range from 0 to 45. However, direction of the weight values (negative or positive) will remain the same, as they reflect which genes are under- and over-expressed in the context of Lyme disease.

Accuracy on the training set was 86.3% (77.7%-92.5%). Misclassification of 3 of 65 control samples and 10 of 30 Lyme samples as seen on FIG. 4B corresponded to a sensitivity of 66.7% and specificity of 95.3% on the training set. The ROC curve (FIG. 4C) had an area under the curve (AUC) of 0.95. This panel of 20 genes was then named the Lyme disease gene expression classifier, and was further tested using the validation set.

TABLE 1-3
Targeted RNA Resequencing Assay Genes

| Gene | GenBank | Gene |
|---|---|---|
| symbol | No. | name |
| ANXA5 | NM_001154 | Annexin A5 |
| ADAMTS10 | NM_030957 | A disintegrin and metalloproteinase |
| | | with thrombospondin motifs 10 |
| ALKBH2 | NM_001145375 | DNA oxidative demethylase ALKBH2 |
| ALPK1 | NM_025144 | Alpha-protein kinase 1 |
| ANPEP | NM_001150 | Aminopeptidase N |
| ARF4 | NM_001660 | ADP-ribosylation factor 4 |
| ARL5B | NM_178815 | ADP-ribosylation |
| | | factor-like protein 5B |
| ASPM | NM_018136 | Abnormal spindle-like |
| | | microcephaly-associated protein |
| AURKA | NM_198433 | Aurora kinase A |
| AZIN1 | NM_015878 | Antizyme inhibitor 1 |
| B4GALT5 | NM_004776 | Beta-1,4-galactosyltransferase 5 |
| BAZ1A | NM_013448 | Bromodomain adjacent to zinc |
| | | finger domain protein 1A |
| BCL6 | NM_001706 | B-cell lymphoma 6 protein |
| BST1 | NM_004334 | ADP-ribosyl cyclase/cyclic |
| | | ADP-ribose hydrolase 2 |
| BTNL8 | NM_024850 | Butyrophilin-like protein 8 |
| BUB1B | NM_001211 | Mitotic checkpoint serine/ |
| | | threonine-protein kinase BUB1 beta |
| C16orf58 | NM_022744 | RUS1 family protein C16orf58 |
| C2orf89 | NM_001080824 | Metalloprotease TIKI1 |
| C3orf14 | NM_020685 | Uncharacterized protein C3orf14 |
| CASC5 | NM_170589 | Protein CASC5 |
| CASP1 | NM_033292 | Caspase-1 |
| CAV1 | NM_001753 | Caveolin-1 |
| CCDC130 | NM_030818 | Coiled-coil domain- |
| | | containing protein 130 |
| CCL20 | NM_004591 | C-C motif chemokine 20 |
| CCNB1 | NM_031966 | G2/mitotic-specific cyclin-B1 |
| CCPG1 | NM_001204451 | Cell cycle progression protein 1 |
| CCR1 | NM_001295 | C-C chemokine receptor type 1 |
| CD300E | NM_181449 | CMRF35-like molecule 2 |
| CD3D | NM_000732 | T-cell surface glycoprotein |
| | | CD3 delta chain |
| CD55 | NM_001114752 | Complement decay- |
| | | accelerating factor |
| CDCA2 | NM_152562 | Cell division cycle- |
| | | associated protein 2 |
| CDCA5 | NM_080668 | Sororin |
| CELF1 | NM_001172639 | CUGBP Elav-like family member 1 |
| CENPF | NM_016343 | Centromere protein F |
| CEP55 | NM_018131 | Centrosomal protein of 55 kDa |
| CKAP4 | NM_006825 | Cytoskeleton-associated protein 4 |
| CLU | NR_045494 | Clustered mitochondria |
| | | protein homolog |
| CR1 | NM_000651 | Clusterin |
| CREB5 | NM_182898 | Complement receptor type 1 |
| CXCL10 | NM_001565 | Cyclic AMP-responsive |
| | | element-binding protein 5 |
| CXCL9 | NM_002416 | C-X-C motif chemokine 10 |
| DEFA5 | NM_021010 | C-X-C motif chemokine 9 |
| DRAM1 | NM_018370 | Defensin-5 |
| DSE | NM_013352 | DNA damage-regulated |
| | | autophagy modulator protein 1 |
| ECT2 | NM_018098 | Dermatan-sulfate epimerase |
| EIF2D | NM_006893 | Protein ECT2 |
| FABP5 | NM_001444 | Eukaryotic translation |
| | | initiation factor 2D |
| FANCI | NM_001113378 | Fatty acid-binding |
| | | protein, epidermal |
| FCAR | NM_133269 | Fanconi anemia group I protein |
| FCGR2A | NM_021642 | Immunoglobulin alpha Fc receptor |
| FDX1L | NM_001031734 | Low affinity immunoglobulin |
| | | gamma Fc region receptor II-a |
| FLT1 | NM_002019 | Adrenodoxin-like protein, |
| | | mitochondrial |
| FPR2 | NM_001005738 | Vascular endothelial growth |
| | | factor receptor 1 |
| GALT | NM_000155 | N-formyl peptide receptor 2 |
| GBP2 | NM_004120 | Galactose-1-phosphate |
| | | uridylyltransferase |
| GBP4 | NM_052941 | Guanylate-binding protein 2 |
| GCA | NM_012198 | Guanylate-binding protein 4 |
| GGT3P | NR_003267 | Grancalcin |
| GLT1D1 | NM_144669 | Putative gamma- |
| | | glutamyltranspeptidase 3 |
| GNG10 | NM_001198664 | Glycosyltransferase 1 domain- |
| | | containing protein 1 |
| GNG5 | NM_005274 | Guanine nucleotide-binding |
| | | protein G(I)/G(S)/G(O) gamma-10 |
| GPR15 | NM_005290 | Guanine nucleotide-binding |
| | | protein G(I)/G(S)/G(O) gamma-5 |
| GPX3 | NM_002084 | G-protein coupled receptor 15 |
| GRAP | NM_006613 | Glutathione peroxidase 3 |
| GRINA | NM_001009184 | GRB2-related adapter protein |
| GRN | NM_002087 | Protein lifeguard 1 |
| HAL | NM_002108 | Granulins |
| HBG2 | NM_000184 | Histidine ammonia-lyase |
| HCAR2 | NM_177551 | Hemoglobin subunit gamma-2 |
| HIST2H2BE | NM_003528 | Hydroxycarboxylic acid receptor 2 |
| HMBS | NM_001024382 | Histone H2B type 2-E |
| HSPA6 | NM_002155 | Porphobilinogen deaminase |
| ICAM1 | NM_000201 | Heat shock 70 kDa protein 6 |
| IFI27 | NM_005532 | Intercellular adhesion molecule 1 |
| IFRD1 | NM_001007245 | Interferon alpha-inducible |
| | | protein 27, mitochondrial |
| IGSF6 | NM_005849 | Interferon-related |
| | | developmental regulator 1 |
| IL23A | NM_016584 | Immunoglobulin superfamily member 6 |
| IL6 | NM_000600 | Interleukin-23 subunit alpha |
| ITGAM | NM_001145808 | Interleukin-6 |
| ITGB7 | NM_000889 | Integrin alpha-M |
| JMJD6 | NM_001081461 | Integrin beta-7 |
| KCNJ2 | NM_000891 | Bifunctional arginine |
| | | demethylase and |
| | | lysyl-hydroxylase JMJD6 |
| KCNMB1 | NM_004137 | Inward rectifier |
| | | potassium channel 2 |
| KIF2C | NM_006845 | Calcium-activated potassium |
| | | channel subunit beta-1 |
| KIF4A | NM_012310 | Kinesin-like protein KIF2C |
| LDLR | NM_001195798 | Chromosome-associated |
| | | kinesin KIF4A |
| LDOC1 | NM_012317 | Low-density lipoprotein |
| | | receptor |
| LIMD2 | NM_030576 | Protein LDOC1 |
| LMNA | NM_170707 | LIM domain-containing protein 2 |
| LOC729737 | NR_039983 | Prelamin-A/C |
| LY9 | NM_002348 | T-lymphocyte surface |
| | | antigen Ly-9 |
| MAP4K1 | NM_007181 | Mitogen-activated protein |
| | | kinase kinase kinase kinase 1 |
| MBOAT2 | NM_138799 | Lysophospholipid |
| | | acyltransferase 2 |
| MIR22HG | NR_028504 | Putative uncharacterized |
| | | protein encoded by MIR22HG |
| MLF1IP | NM_024629 | Centromere protein U |
| MLLT6 | NM_005937 | Protein AF-17 |
| MSI2 | NM_138962 | RNA-binding protein |
| | | Musashi homolog 2 |
| MXD1 | NM_002357 | Max dimerization protein 1 |
| MYBL2 | NM_002466 | Myb-related protein B |
| NANS | NM_018946 | Sialic acid synthase |
| NCF1 | NM_000265 | Neutrophil cytosol factor 1 |
| NIF3L1 | NM_021824 | NIF3-like protein 1 |
| NR3C2 | NM_000901 | Mineralocorticoid receptor |
| NUSAP1 | NM_018454 | Nucleolar and spindle- |
| | | associated protein 1 |
| OAS2 | NM_016817 | 2′-5′-oligoadenylate |
| | | synthase 2 |
| OMG | NM_002544 | Oligodendrocyte-myelin |
| | | glycoprotein |
| ORC1 | NM_004153 | Origin recognition |
| | | complex subunit 1 |
| OXSR1 | NM_005109 | Serine/threonine-protein kinase OSR1 |
| PABPC3 | NM_030979 | Polyadenylate-binding protein 3 |
| PECAM1 | NM_000442 | Platelet endothelial |
| | | cell adhesion molecule |
| PHF15 | NM_015288 | Protein Jade-2 |
| PIK3R2 | NM_005027 | Phosphatidylinositol 3-kinase |
| | | regulatory subunit beta |
| PKD1P1 | NR_036447 | Polycystin 1, transient receptor |
| | | potential channel interacting |
| | | pseudogene 1 |
| PLBD1 | NM_024829 | Phospholipase B-like 1 |
| PLK1 | NM_005030 | Serine/threonine- |
| | | protein kinase PLK1 |
| PNPLA1 | NM_173676 | Patatin-like phospholipase |
| | | domain-containing protein 1 |
| POMT1 | NM_007171 | Protein O-mannosyl- |
| | | transferase 1 |
| PSME1 | NM_006263 | Proteasome activator complex subunit 1 |
| QPCT | NM_012413 | Glutaminyl-peptide |
| | | cyclotransferase |
| RAB12 | NM_001025300 | Ras-related protein Rab-12 |
| RAD51 | NM_133487 | DNA repair protein |
| | | RAD51 homolog 1 |
| RBMX | NR_028477 | RNA-binding motif |
| | | protein, X chromosome |
| RPL11 | NM_001199802 | 60S ribosomal protein L11 |
| RPL29 | NM_000992 | 60S ribosomal protein L29 |
| RPL6 | NM_001024662 | 60S ribosomal protein L6 |
| RPS5 | NM_001009 | 40S ribosomal protein S5 |
| RRM2 | NM_001165931 | Ribonucleoside-diphosphate |
| | | reductase subunit M2 |
| SAMSN1 | NM_001256370 | SAM domain-containing |
| | | protein SAMSN-1 |
| SERPINA1 | NM_001127705 | Alpha-1-antitrypsin |
| SERPING1 | NM_000062 | Plasma protease C1 inhibitor |
| SETD5 | NM_001080517 | SET domain- |
| | | containing protein 5 |
| SHCBP1 | NM_024745 | SHC SH2 domain- |
| | | binding protein 1 |
| SIGLEC5 | NM_003830 | Sialic acid-binding |
| | | Ig-like lectin 5 |
| SIRPA | NM_080792 | Tyrosine-protein phosphatase |
| | | non-receptor type substrate 1 |
| SIRPD | NM_178460 | Signal-regulatory protein delta |
| SLC15A3 | NM_016582 | Solute carrier |
| | | family 15 member 3 |
| SLC25A37 | NM_016612 | Mitoferrin-1 |
| SLC31A2 | NM_001860 | Probable low affinity |
| | | copper uptake protein 2 |
| SNRNP27 | NR_037862 | U4/U6.U5 small nuclear |
| | | ribonucleoprotein 27 kDa protein |
| SOCS3 | NM_003955 | Suppressor of cytokine signaling 3 |
| SORT1 | NM_002959 | Sortilin |
| SPAG5 | NM_006461 | Sperm-associated antigen 5 |
| STAB1 | NM_015136 | Stabilin-1 |
| STAT1 | NM_007315 | Signal transducer and activator |
| | | of transcription 1-alpha/beta |
| STEAP4 | NM_001205315 | Metalloreductase STEAP4 |
| STMN3 | NM_015894 | Stathmin-3 |
| SYTL1 | NM_032872 | Synaptotagmin-like protein 1 |
| TBCCD1 | NM_018138 | TBCC domain-containing protein 1 |
| TBP | NM_003194 | TATA-box-binding protein |
| TCEB1 | NM_001204861 | Transcription elongation |
| | | factor B polypeptide 1 |
| TJP2 | NM_004817 | Tight junction protein ZO-2 |
| TLR2 | NM_003264 | Toll-like receptor 2 |
| TNFRSF10C | NM_003841 | Tumor necrosis |
| | | factor receptor |
| | | superfamily member 10C |
| TNFSF10 | NM_003810 | Tumor necrosis |
| | | factor ligand |
| | | superfamily member 10 |
| TNFSF13B | NM_006573 | Tumor necrosis |
| | | factor ligand |
| | | superfamily member 13B |
| TP53I13 | NM_138349 | Tumor protein p53- |
| | | inducible protein 13 |
| TPM4 | NM_001145160 | Tropomyosin alpha-4 chain |
| TPX2 | NM_012112 | Targeting protein for Xklp2 |
| TREM1 | NM_018643 | Triggering |
| | | receptor expressed |
| | | on myeloid cells 1 |
| TTK | NM_003318 | Dual specificity |
| | | protein kinase TTK |
| TXNDC5 | NM_030810 | Thioredoxin domain- |
| | | containing protein 5 |
| TYMP | NM_001953 | Thymidine phosphorylase |
| TYMS | NM_001071 | Thymidylate synthase |
| UBE2J1 | NM_016021 | Ubiquitin-conjugating |
| | | enzyme E2 J1 |
| VASP | NM_003370 | Vasodilator- |
| | | stimulated phosphoprotein |
| VMP1 | NM_030938 | Vacuole membrane protein 1 |
| WARS | NM_173701 | Tryptophan--tRNA |
| | | ligase, cytoplasmic |
| WDR85 | NM_138778 | Diphthine methyltransferase |
| ZFP161 | NM_001243704 | Zinc finger and BTB |
| | | domain-containing protein 14 |
| ZNF276 | NM_152287 | Zinc finger protein 276 |
| ZNF384 | NM_001135734 | Zinc finger protein 384 |
| ZNF549 | NM_001199295 | Zinc finger protein 549 |

TABLE 1-4
Lyme Disease Diagnostic Panel Genes

| Gene symbol | 1st gene panel | 2nd gene panel | 3rd gene panel |
|---|---|---|---|
| ANXA5 | yes | yes | yes |
| ADAMTS10 | yes | — | — |
| ALKBH2 | yes | — | — |
| ALPK1 | yes | — | — |
| ANPEP | yes | yes | — |
| ARF4 | yes | — | — |
| ARL5B | yes | — | — |
| ASPM | yes | yes | — |
| AURKA | yes | — | — |
| AZIN1 | yes | yes | — |
| B4GALT5 | yes | — | — |
| BAZ1A | yes | — | — |
| BCL6 | yes | — | — |
| BST1 | yes | yes | — |
| BTNL8 | yes | — | — |
| BUB1B | yes | yes | — |
| C16orf58 | yes | — | — |
| C2orf89 | yes | — | — |
| C3orf14 | yes | yes | yes |
| CASC5 | yes | yes | — |
| CASP1 | yes | yes | — |
| CAV1 | yes | yes | — |
| CCDC130 | yes | yes | — |
| CCL20 | yes | — | — |
| CCNB1 | yes | yes | — |
| CCPG1 | yes | — | — |
| CCR1 | yes | — | — |
| CD300E | yes | — | — |
| CD3D | yes | yes | — |
| CD55 | yes | yes | — |
| CDCA2 | yes | yes | yes |
| CDCA5 | yes | yes | — |
| CELF1 | yes | — | — |
| CENPF | yes | yes | — |
| CEP55 | yes | yes | — |
| CKAP4 | yes | yes | — |
| CLU | yes | — | — |
| CR1 | yes | yes | yes |
| CREB5 | yes | — | — |
| CXCL10 | yes | yes | — |
| CXCL9 | yes | yes | — |
| DEFA5 | yes | yes | — |
| DRAM1 | yes | yes | — |
| DSE | yes | — | — |
| ECT2 | yes | yes | — |
| EIF2D | yes | yes | — |
| FABP5 | yes | yes | — |
| FANCI | yes | yes | — |
| FCAR | yes | — | — |
| FCGR2A | yes | — | — |
| FDX1L | yes | yes | — |
| FLT1 | yes | — | — |
| FPR2 | yes | yes | — |
| GALT | yes | — | — |
| GBP2 | yes | yes | yes |
| GBP4 | yes | yes | — |
| GCA | yes | — | — |
| GGT3P | yes | — | — |
| GLT1D1 | yes | — | — |
| GNG10 | yes | — | — |
| GNG5 | yes | — | — |
| GPR15 | yes | yes | — |
| GPX3 | yes | yes | — |
| GRAP | yes | — | — |
| GRINA | yes | — | — |
| CRN | yes | yes | — |
| HAL | yes | — | — |
| HBG2 | yes | — | — |
| HCAR2 | yes | — | — |
| HIST2H2BE | yes | — | — |
| HMBS | yes | yes | — |
| HSPA6 | yes | — | — |
| ICAM1 | yes | yes | — |
| IFI27 | yes | yes | yes |
| IFRD1 | yes | yes | — |
| IGSF6 | yes | yes | — |
| IL23A | yes | — | — |
| IL6 | yes | — | — |
| ITGAM | yes | yes | yes |
| ITGB7 | yes | yes | — |
| JMJD6 | yes | yes | — |
| KCNJ2 | yes | yes | yes |
| KCNMB1 | yes | — | — |
| KIF2C | yes | yes | — |
| KIF4A | yes | yes | yes |
| LDLR | yes | yes | — |
| LDOC1 | yes | — | — |
| LIMD2 | yes | — | — |
| LMNA | yes | yes | — |
| LOC729737 | yes | — | — |
| LY9 | yes | — | — |
| MAP4K1 | yes | — | — |
| MBOAT2 | yes | — | — |
| MIR22HG | yes | — | — |
| MLF1IP | yes | yes | yes |
| MLLT6 | yes | — | — |
| MSI2 | yes | — | — |
| MXD1 | yes | yes | — |
| MYBL2 | yes | yes | — |
| NANS | yes | — | — |
| NCF1 | yes | yes | yes |
| NIF3L1 | yes | yes | — |
| NR3C2 | yes | — | — |
| NUSAP1 | yes | yes | — |
| OAS2 | yes | yes | — |
| OMG | yes | — | — |
| ORC1 | yes | yes | — |
| OXSR1 | yes | — | — |
| PABPC3 | yes | — | — |
| PECAM1 | yes | — | — |
| PHF15 | yes | — | — |
| PIK3R2 | yes | — | — |
| PKD1P1 | yes | — | — |
| PLBD1 | yes | yes | yes |
| PLK1 | yes | yes | yes |
| PNPLA1 | yes | — | — |
| POMT1 | yes | yes | — |
| PSME1 | yes | yes | — |
| QPCT | yes | — | — |
| RAB12 | yes | yes | — |
| RAD51 | yes | yes | yes |
| RBMX | yes | — | — |
| RPL11 | yes | — | — |
| RPL29 | yes | — | — |
| RPL6 | yes | — | — |
| RPS5 | yes | — | — |
| RRM2 | yes | yes | — |
| SAMSN1 | yes | — | — |
| SERPINA1 | yes | — | — |
| SERPING1 | yes | — | — |
| SETD5 | yes | — | — |
| SHCBP1 | yes | yes | — |
| SIGLEC5 | yes | — | — |
| SIRPA | yes | — | — |
| SIRPD | yes | yes | — |
| SLC15A3 | yes | — | — |
| SLC25A37 | yes | yes | yes |
| SLC31A2 | yes | — | — |
| SNRNP27 | yes | — | — |
| SOCS3 | yes | yes | — |
| SORT1 | yes | yes | — |
| SPAG5 | yes | yes | — |
| STAB1 | yes | yes | yes |
| STAT1 | yes | yes | — |
| STEAP4 | yes | yes | yes |
| STMN3 | yes | — | — |
| SYTL1 | yes | yes | — |
| TBCCD1 | yes | — | — |
| TBP | yes | yes | yes |
| TCEB1 | yes | — | — |
| TJP2 | yes | — | — |
| TLR2 | yes | yes | — |
| TNFRSF10C | yes | — | — |
| TNFSF10 | yes | yes | — |
| TNFSF13B | yes | yes | yes |
| TP53I13 | yes | — | — |
| TPM4 | yes | — | — |
| TPX2 | yes | yes | — |
| TREM1 | yes | yes | — |
| TTK | yes | yes | — |
| TXNDC5 | yes | — | — |
| TYMP | yes | yes | — |
| TYMS | yes | yes | — |
| UBE2J1 | yes | — | — |
| VASP | yes | — | — |
| VMP1 | yes | — | — |
| WARS | yes | yes | — |
| WDR85 | yes | — | — |
| ZFP161 | yes | yes | — |
| ZNF276 | yes | yes | yes |
| ZNF384 | yes | yes | — |
| ZNF549 | yes | — | — |

TABLE 1-5
Lyme Disease Classifier Genes

| Gene | Gene | | |
|---|---|---|---|
| symbol | name | Weight | Rank |
| (Intercept) | NA | −5.72E−01 | NA |
| ANXA5 | Annexin A5 | 4.40E−03 | 2 |
| C3orf14 | Uncharacterized | −9.73E−03 | 16 |
| | protein C3orf14 | | |
| CDCA2 | Cell division cycle- | −4.34E−03 | 6 |
| | associated protein 2 | | |
| CR1 | Complement receptor type 1 | −2.26E−03 | 3 |
| GBP2 | Guanylate-binding protein 2 | 6.43E−04 | 9 |
| IFI27 | Interferon alpha-inducible | −6.97E−05 | 15 |
| | protein 27, mitochondrial | | |
| ITGAM | Integrin alpha-M | −3.26E−03 | 13 |
| KCNJ2 | Inward rectifier | −9.01E−03 | 10 |
| | potassium channel 2 | | |
| KIF4A | Chromosome-associated | 3.82E−03 | 12 |
| | kinesin KIF4A | | |
| MLF1IP | Centromere protein U | −1.09E−02 | 5 |
| NCF1 | Neutrophil cytosol | −7.56E−04 | 1 |
| | factor 1 | | |
| PLBD1 | Phospholipase | −2.36E−04 | 19 |
| | B-like 1 | | |
| PLK1 | Serine/threonine- | 1.35E−03 | 18 |
| | protein kinase PLK1 | | |
| RAD51 | DNA repair protein | 6.75E−02 | 14 |
| | RAD51 homolog 1 | | |
| SLC25A37 | Mitoferrin-1 | 1.89E−04 | 20 |
| STAB1 | Stabilin-1 | −1.51E−03 | 4 |
| STEAP4 | Metalloreductase STEAP4 | 3.64E−03 | 17 |
| TBP | TATA-box-binding protein | 1.67E−02 | 11 |
| TNFSF13B | Tumor necrosis factor | 2.48E−03 | 7 |
| | ligand superfamily member 13B | | |
| ZNF276 | Zinc finger protein 276 | −7.33E−03 | 8 |

On the validation set, the Lyme disease gene expression classifier (20 gene panel) scored an accuracy of 91.6% (95%[84.1%-96.3%]) based on a 93.3% sensitivity and 90.8% specificity, from misclassifying 6 or 65 control samples and 2 of 30 Lyme samples (FIG. 4D). The ROC curve (FIG. 4E) had an area under the curve (AUC) of 0.92. The kappa statistic was 0.812, the positive predictive value was 0.967, and the negative predictive value was 0.824. Almost all of the seropositive Lyme samples were correctly identified; 17 of 18 (94.4%) samples from patients who were Lyme seropositive at the first doctor visit, and 9 of 10 (90%) samples from patients who seroconverted after the first visit were correctly classified as Lyme. The algorithm also classified 16 of 30 (53.3%) samples from seronegative Lyme disease patients as Lyme (FIG. 4F).

Representative gene expression values shown as read counts from targeted RNA expression resequencing are provided in Table 1-6. Representative weighted gene expression values are provided in Table 1-7A and Table 1-7B.

TABLE 1-6
Representative Gene Expression Values\hat{ }

| | Subject | | | | | | | |
|---|---|---|---|---|---|---|---|---|
| Gene | Lyme 1 | Lyme 2 | Healthy 1 | Healthy 2 | Healthy 3 | Bac | Flu | TB |
| ANXA5 | 354.09 | 345.55 | 69.82 | 174.85 | 115.1 | 232.88 | 168.06 | 87.67 |
| C3orf14 | 14.18 | 2.25 | 8.22 | 20.1 | 0.1 | 4.21 | 12.29 | 16.63 |
| CDCA2 | 0.11 | 1.88 | 0 | 0.63 | 0.7 | 6.01 | 17.55 | 1.58 |
| CR1 | 40.15 | 58.97 | 48.51 | 41.67 | 55.66 | 105.72 | 25.45 | 61.99 |
| GBP2 | 283.21 | 377.43 | 317.23 | 211.91 | 368.14 | 306.11 | 518.23 | 372.39 |
| IF127 | 0 | 1.45 | 6.38 | 0.63 | 114.35 | 170.16 | 7.46 | 23.98 |
| ITGAM | 155.51 | 160.17 | 92.32 | 71.2 | 83.19 | 115.98 | 56.17 | 58.48 |
| KCNJ2 | 4.33 | 0 | 6.88 | 23.45 | 19.81 | 18.12 | 110.14 | 18.21 |
| KIF4A | 186.08 | 30.42 | 0 | 2.72 | 0 | 2.68 | 0 | 4.19 |
| MLF1IP | 5.74 | 17.87 | 3.86 | 10.89 | 10.6 | 28.98 | 0.88 | 9.73 |
| NCF1 | 296.84 | 204.06 | 1559.8 | 257.56 | 346.63 | 556.74 | 231.69 | 899.3 |
| PLBD1 | 367.51 | 323.72 | 830.51 | 1419.96 | 234.52 | 466.46 | 546.32 | 1330.97 |
| PLK1 | 14.83 | 75.07 | 14.77 | 18.22 | 6.92 | 32.82 | 0.88 | 15.84 |
| RAD51 | 0 | 0 | 0.67 | 1.47 | 0.65 | 0.55 | 0 | 0 |
| SLC25A37 | 121.8 | 310.84 | 109.94 | 72.87 | 1130.65 | 485.74 | 186.93 | 358.14 |
| STAB1 | 0 | 8.05 | 7.72 | 3.56 | 0.8 | 5.41 | 0.44 | 1.58 |
| STEAP4 | 417.39 | 91.22 | 132.6 | 55.28 | 21.16 | 24.64 | 74.6 | 140.95 |
| TBP | 32.41 | 86.6 | 3.52 | 14.03 | 0.15 | 12.02 | 3.51 | 2.49 |
| TNFSF13B | 49.08 | 64.76 | 32.06 | 89.41 | 12 | 29.31 | 12.29 | 55.77 |
| ZNF276 | 52.05 | 110.53 | 80.9 | 156.21 | 14.79 | 49.69 | 20.62 | 28.51 |

\hat{ }Abbreviations: Bac (bacteremia); Flu (influenza); and TB (tuberculosis).

TABLE 1-7A
Weighted Gene Expression Values for Lyme Disease and Healthy Subjects*

| Gene | Weight | Lyme 1 | Lyme 2 | Healthy 1 | Healthy 2 | Healthy 3 |
|---|---|---|---|---|---|---|
| intercept | −0.572 | −0.572 | −0.572 | −0.572 | −0.572 | −0.572 |
| ANXA5 | 0.0044 | 1.557996 | 1.52042 | 0.307208 | 0.76934 | 0.50644 |
| C3orf14 | −0.00973 | −0.1379714 | −0.0218925 | −0.0799806 | −0.195573 | −0.000973 |
| CDCA2 | −0.00434 | −0.0004774 | −0.0081592 | 0 | −0.0027342 | −0.003038 |
| CR1 | −0.00226 | −0.090739 | −0.1332722 | −0.1096326 | −0.0941742 | −0.1257916 |
| GBP2 | 0.000643 | 0.18210403 | 0.24268749 | 0.20397889 | 0.13625813 | 0.23671402 |
| IF127 | −0.0000697 | 0 | −0.00010107 | −0.00044469 | −0.00004391 | −0.00797020 |
| ITGAM | −0.00326 | −0.5069626 | −0.5221542 | −0.3009632 | −0.232112 | −0.2711994 |
| KCNJ2 | −0.00901 | −0.0390133 | 0 | −0.0619888 | −0.2112845 | −0.1784881 |
| KIF4A | 0.00382 | 0.7108256 | 0.1162044 | 0 | 0.0103904 | 0 |
| MLF1IP | −0.0109 | −0.062566 | −0.194783 | −0.042074 | −0.118701 | −0.11554 |
| NCF1 | −0.000756 | −0.22441104 | −0.15426936 | −1.1792088 | −0.19471536 | −0.26205228 |
| PLBD1 | −0.000236 | −0.08673236 | −0.07639792 | −0.19600036 | −0.33511056 | −0.05534672 |
| PLK1 | 0.00135 | 0.0200205 | 0.1013445 | 0.0199395 | 0.024597 | 0.009342 |
| RAD51 | 0.0675 | 0 | 0 | 0.045225 | 0.099225 | 0.043875 |
| SLC25A37 | 0.000189 | 0.0230202 | 0.05874876 | 0.02077866 | 0.01377243 | 0.21369285 |
| STAB1 | −0.00151 | 0 | −0.0121555 | −0.0116572 | −0.0053756 | −0.001208 |
| STEAP4 | 0.00364 | 1.5192996 | 0.3320408 | 0.482664 | 0.2012192 | 0.0770224 |
| TBP | 0.0167 | 0.541247 | 1.44622 | 0.058784 | 0.234301 | 0.002505 |
| TNFSF13B | 0.00248 | 0.1217184 | 0.1606048 | 0.0795088 | 0.2217368 | 0.02976 |
| ZNF276 | −0.00733 | −0.3815265 | −0.8101849 | −0.592997 | −1.1450193 | −0.1084107 |

RAW LYME DISEASE SCORE

| | | | | |
|---|---|---|---|---|
| 2.57383173 | 1.472900905 | −1.92886040 | −1.39600367 | −0.58266673 |

SCALED LYME DISEASE SCORE

| | | | | |
|---|---|---|---|---|
| 0.92899576 | 0.81296671 | 0.1268622 | 0.19828005 | 0.35829639 |

*Rounded to the nearest 1 × 10⁻⁸ for readability.

TABLE 1-7B
Weighted Gene Expression Values for Lyme Disease and Control Subjects*

| Gene | Weight | Lyme 1 | Lyme 2 | Bac | Flu | TB |
|---|---|---|---|---|---|---|
| intercept | −0.572 | −0.572 | −0.572 | −0.572 | −0.572 | −0.572 |
| ANXA5 | 0.0044 | 1.557996 | 1.52042 | 1.024672 | 0.739464 | 0.385748 |
| C3orf14 | −0.00973 | −0.1379714 | −0.0218925 | −0.0409633 | −0.1195817 | −0.1618099 |
| CDCA2 | −0.00434 | −0.0004774 | −0.0081592 | −0.0260834 | −0.076167 | −0.0068572 |
| CR1 | −0.00226 | −0.090739 | −0.1332722 | −0.2389272 | −0.057517 | −0.1400974 |
| GBP2 | 0.000643 | 0.18210403 | 0.24268749 | 0.19682873 | 0.33322189 | 0.23944677 |
| IF127 | −0.0000697 | 0 | −0.00010107 | −0.01186015 | −0.00051996 | −0.00167141 |
| ITGAM | −0.00326 | −0.5069626 | −0.5221542 | −0.3780948 | −0.1831142 | −0.1906448 |
| KCNJ2 | −0.00901 | −0.0390133 | 0 | −0.1632612 | −0.9923614 | −0.1640721 |
| KIF4A | 0.00382 | 0.7108256 | 0.1162044 | 0.0102376 | 0 | 0.0160058 |
| MLF1IP | −0.0109 | −0.062566 | −0.194783 | −0.315882 | −0.009592 | −0.106057 |
| NCF1 | −0.000756 | −0.22441104 | −0.15426936 | −0.42089544 | −0.17515764 | −0.6798708 |
| PLBD1 | −0.000236 | −0.08673236 | −0.07639792 | −0.11008456 | −0.12893152 | −0.31410892 |
| PLK1 | 0.00135 | 0.0200205 | 0.1013445 | 0.044307 | 0.001188 | 0.021384 |
| RAD51 | 0.0675 | 0 | 0 | 0.037125 | 0 | 0 |
| SLC25A37 | 0.000189 | 0.0230202 | 0.05874876 | 0.09180486 | 0.03532977 | 0.06768846 |
| STAB1 | −0.00151 | 0 | −0.0121555 | −0.0081691 | −0.0006644 | −0.0023858 |
| STEAP4 | 0.00364 | 1.5192996 | 0.3320408 | 0.0896896 | 0.271544 | 0.513058 |
| TBP | 0.0167 | 0.541247 | 1.44622 | 0.200734 | 0.058617 | 0.041583 |
| TNFSF13B | 0.00248 | 0.1217184 | 0.1606048 | 0.0726888 | 0.0304792 | 0.1383096 |
| ZNF276 | −0.00733 | −0.3815265 | −0.8101849 | −0.3642277 | −0.1511446 | −0.2089783 |

RAW LYME DISEASE SCORE

| | | | | |
|---|---|---|---|---|
| 2.57383173 | 1.472900905 | -0.88236126 | -0.99690756 | -1.12533 |

SCALED LYME DISEASE SCORE

| | | | | |
|---|---|---|---|---|
| 0.92899576 | 0.81296671 | 0.29265177 | 0.26946634 | 0.24499452 |

*Abbreviations: Bac (bacteremia); Flu (influenza); and TB (tuberculosis).
Rounded to the nearest 1 × 10⁻⁸ for readability

Various modifications and variations of the present disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are understood by those skilled in the art are intended to be within the scope of the claims.

1. A method for measuring gene expression, comprising the steps of:
(a) measuring RNA expression of a plurality of genes of cells from a blood sample obtained from a mammalian subject suspected of having a tick-borne disease;
(b) calculating a weighted RNA expression score for each of the plurality of genes; and
(c) calculating a Lyme disease score by taking the sum of the weighted RNA expression scores.

2. The method of claim 1 for providing information to assess whether a subject has acute Lyme disease, further comprising: step (d) identifying the subject as not having acute Lyme disease when the Lyme disease score is negative; or identifying the subject as having acute Lyme disease when the Lyme disease score is positive.

3. The method of claim 1, further comprising: obtaining a blood sample from the subject prior to step (a).

4. The method of claim 1, wherein the cells are peripheral blood mononuclear cells (PBMCs) isolated from the blood sample.

5. The method of claim 4, further comprising: extracting RNA from the PBMCs prior to step (a).

6. The method of claim 1, wherein the plurality of genes comprises at least 3, 4, 5, 6, 7, 8, 9, 10, 11, 12, 13, 14, 15, 16, 17, 18, 19 or all 20 genes of the group consisting of ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276.

7. The method of claim 6, wherein the plurality of genes comprises NCF1.

8. The method of claim 6, wherein the plurality of genes comprises ANXA5.

9. The method of claim 6, wherein the plurality of genes comprises CR1.

10. The method of claim 6, wherein the plurality of genes comprises STAB1.

11. The method of claim 6, wherein the plurality of genes comprises MLF1IP.

12. The method of claim 1, wherein step (a) comprises one or more of the group consisting of sequence analysis, hybridization, and amplification.

13. The method of any one of claims 4 to 12, wherein step (a) comprises targeted RNA expression resequencing comprising:
(i) preparing an RNA expression library for the plurality of targeted genes from RNA extracted from the PBMCs;
(ii) sequencing a portion of at least 50,000 members of the library; and
(iii) generating a read count for RNA expression of the plurality of genes by normalization to the sequence of the at least 50,000 members of step (ii).

14. The method of any one of claims 4 to 12, wherein step (a) comprises whole transcriptome shotgun sequencing (WTSS) comprising:
(i) preparing an RNA expression library for the plurality of genes from RNA extracted from the PBMCs;
(ii) sequencing a portion of at least 1,000,000 members of the library; and
(iii) generating a read count for RNA expression of the plurality of genes by normalization to the sequence of the at least 1,000,000 members of step (ii).

15. The method of claim 1, wherein step (b) comprises: multiplying the read count for each of the plurality of genes by a predetermined gene expression weight to obtain the weighted RNA expression score.

16. The method of any one claims 4 to 12, wherein step (a) comprises: performing reverse transcriptase-quantitative polymerase chain reaction (RT-qPCR) on RNA extracted from the PBMCs.

17. The method of any one claims 4 to 12, wherein step (a) comprises: hybridizing RNA extracted from the PBMCs to a microarray.

18. The method of any one claims 4 to 12, wherein step (a) comprises: performing serial amplification of gene expression (SAGE) on RNA extracted from the PBMCs.

19. The method of claim 1, wherein the subject was bitten by a tick in a region where at least 20% of ticks are suspected of being infected with Borrelia burgdorferi.

20. The method of claim 1, wherein the subject was bitten by a tick within three weeks of the blood sample being obtained.

21. The method of claim 1, wherein the subject has an erythema migrans rash when the blood sample was obtained.

22. The method of claim 1, wherein the subject does not have an erythema migrans rash when the blood sample was obtained.

23. The method of claim 21 or claim 22, wherein the subject has flu-like symptoms when the blood sample was obtained.

24. The method of claim 1, further comprising performing a serologic test for Lyme disease.

25. The method of claim 24, wherein the subject was determined to be negative for Lyme disease by serologic testing at the time the blood sample was obtained.

26. The method of claim 1, wherein the tick-borne disease is selected from the group consisting of Borreliosis, Southern tick associated rash illness, Q fever, Colorado tick fever, Powassan virus infection, tick-borne encephalitis virus infection, tick-borne relapsing fever, Heartland virus infection and severe fever with thrombocytopenia virus infection.

27. The method of claim 2, further comprising:
step (e) administering an antibiotic therapy to the subject to treat the Lyme disease when the subject has been identified as having acute Lyme disease.

28. The method of claim 27, wherein the antibiotic therapy comprises an effective amount of an antibiotic selected from the group consisting of tetracyclines, penicillins, and cephalosporins.

29. The method of claim 27, wherein the antibiotic therapy comprises an oral regimen comprising doxycycline, amoxicillin or cefuroxime axetil.

30. The method of claim 27, wherein the antibiotic therapy comprises a parenteral regimen comprising ceftriaxone, cefotaxime, or penicillin G.

31. A kit comprising:
(a) a plurality of oligonucleotides which hybridize to a plurality of genes comprising at least 2, 3, 4, 5, 6, 7, 8, 9, 10, 11, 12, 13, 14, 15, 16, 17, 18, 19 or all 20 genes of the group consisting of ANXA5, C3orf14, CDCA2, CR1, GBP2, IFI27, ITGAM, KCNJ2, KIF4A, MLF1IP, NCF1, PLBD1, PLK1, RAD51, SLC25A37, STAB1, STEAP4, TBP, TNFSF13B, and ZNF276; and
(b) instructions for: (i) use of the oligonucleotides for measuring RNA expression of the plurality of genes; (ii) calculating a weighted RNA expression score for each of the plurality of genes; and (iii) calculating a Lyme disease score by taking the sum of the weighted RNA expression scores.

32. The method of claim 31, wherein the plurality of genes comprises NCF1.

33. The method of claim 31, wherein the plurality of genes comprises ANXA5.

34. The method of claim 31, wherein the plurality of genes comprises CR1.

35. The method of claim 31, wherein the plurality of genes comprises STAB1.

36. The method of claim 31, wherein the plurality of genes comprises MLF1IP.
